(19) United States
(12) Patent Application Publication
(10) Pub. No.: US 2014/0147510 A1
(43) Pub. Date: May 29, 2014

(54) MULTIPHASIC POLYMERIC PARTICLES CAPABLE OF SHAPE-SHIFTING VIA ENVIRONMENTAL STIMULATION

(71) Applicants: Joerg Lahann, Ann Arbor, MI (US); Jaewon Yoon, Ann Arbor, MI (US); Srijanani Bhaskar, Boothwyn, PA (US); Kyungjin Lee, Daejeon (KR)

(72) Inventors: Joerg Lahann, Ann Arbor, MI (US); Jaewon Yoon, Ann Arbor, MI (US); Srijanani Bhaskar, Boothwyn, PA (US); Kyungjin Lee, Daejeon (KR)

(73) Assignee: THE REGENTS OF THE UNIVERSITY OF MICHIGAN, Ann Arbor, MI (US)

(21) Appl. No.: 13/882,109

(22) PCT Filed: Oct. 27, 2011
(86) PCT No.: PCT/US11/58142

§ 371 Date: Aug. 20, 2013

Related U.S. Application Data
(60) Provisional application No. 61/407,793, filed on Oct. 28, 2010.

(51) Int. Cl. A61K 9/14

(52) U.S. Cl. CPC A61K 9/14
USPC 424/501; 514/772.3; 514/772.6; 264/340

(57) ABSTRACT

Provided herein are methods of making and controlling multiphasic polymeric micro-components capable of shape-shifting. Such a multiphasic micro-component comprises a first phase (that can include a first polymer) and at least one additional phase distinct from said first phase (that can include a second polymer). One or more of the first phase and additional phase comprises a component that is responsive to an external stimulus. Thus, the micro-component exhibits a substantial physical deformation in response to: (i) the presence of the external stimulus or (ii) a change in the external stimulus. Exemplary external stimuli include temperature, pressure, light, pH, ionic strength, hydrophobicity/hydrophilicity, solvent, concentration, a stimulator chemical, sonic energy, electric energy, pressure, magnetic fields, and combinations thereof.

### CROSS-REFERENCE TO RELATED APPLICATIONS

This application claims the benefit of U.S. Provisional Application No. 61/407,793, filed on Oct. 28, 2010. The entire disclosure of the above application is incorporated herein by reference.

### FIELD

The present disclosure relates to methods of shape-shifting multiphasic polymeric particles via environmental stimulation and to the multiphasic polymeric particles capable of such shape-shifting.

### BACKGROUND

This section provides background information related to the present disclosure which is not necessarily prior art.

Control of anisotropic particles with specific size, shape, and functionality is important in many technological fields. In particular, control of anisotropic particles is important for bioapplications, such as biosensor or drug delivery system. It is desirable to have the potential to control polymeric particles, for example, by controlling particle functionalities, especially when they possess “stimuli responsive” or “environmentally sensitive” features. In accordance with the present disclosure, new methods are provided for fabrication and control of multiphasic particles that can be shape-shifted upon presentation of an external stimulus, for example, in the form of physical or chemical environmental changes. Thus, the present teachings provide dynamic control over anisotropic particles. Further, the present disclosure also provides multiphasic particles capable of shape-shifting, providing the ability to control particle size and shape with environmental condition due to site-specific properties of internal phases.

### SUMMARY

This section provides a general summary of the disclosure, and is not a comprehensive disclosure of its full scope or all of its features.

In various aspects, the present teachings provide multiphasic (e.g., multicompartmental) micro-components capable of shape-shifting or shape-toggling. In certain aspects, the present disclosure provides a multiphasic micro-component comprising a first phase and at least one additional phase distinct from the first phase. One or more of the first phase and the at least one additional phase comprises a polymer. Further, one or more of the first phase and the additional phase comprises a component that is responsive to an external stimulus so that the micro-component exhibits a substantial physical deformation in response to either: (i) the presence of the external stimulus or (ii) a change in level of the external stimulus. In certain aspects, the component that is responsive to an external stimulus is the polymer in the first phase. In certain aspects, the one or more additional phases also comprise one or more components that are responsive to an external stimulus. In certain variations, the first phase and the additional phases may be responsive to the same stimulus, while in alternative variations, the first phase may be responsive to a first external stimulus, while the additional phase(s) are responsive to a second distinct external stimulus.

In other aspects, the present teachings provide methods of controlling the shape of multiphasic (e.g., multicompartmental) microcomponents. In one aspect, such a method of controlling the shape of a micro-component comprises exposing a multiphasic micro-component capable of substantial deformation to an external stimulus. The multiphasic micro-component comprises a first phase and at least one additional phase distinct from the first phase. At least one of the first phase and the at least one additional phase comprises a polymer. Further, at least one of the first phase and the at least one additional phase comprises a component that is responsive to the external stimulus. The at least one additional phase exhibits a substantial deformation resulting in a change in shape, volume, or both shape and volume.

In other variations, the present disclosure provides an alternative multiphasic micro-component capable of shape-shifting. The micro-component comprises a first phase comprising a first polymer responsive to an external stimulus and at least one additional phase distinct from the first phase comprising a second polymer distinct from the first polymer. The first phase exhibits a substantial physical deformation in response to: (i) the presence of the external stimulus or (ii) a change in the external stimulus. The first and second polymers are independently selected from the group consisting of: poly(lactide-co-glycolide) (PLGA), poly(vinyl cinnamate) (PVCi), poly(methyl methacrylate) (PMMA), poly(ethylene) oxide (PEO), and combinations thereof.

In yet other variations, a multiphasic micro-component is provided that is capable of shape-toggling. For example, the micro-component optionally comprises a first phase comprising a first polymer responsive to a first external stimulus. The micro-component also comprises at least one additional phase distinct from the first phase comprising a second polymer distinct from the first polymer and responsive to a second external stimulus. The first phase exhibits a substantial physical deformation in response to: (i) the presence of the first external stimulus or (ii) a change in the first external stimulus. The additional phase exhibits a substantial physical deformation in response to: (i) the presence of the second external stimulus or (ii) a change in the second external stimulus.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1: A-B shows confocal laser scanning microscopy (CLSM) and scanning electron microscopy (SEM) micrographs of a biphasic/bicompartmental poly(lactide-co-glycolide) (PLGA) micro-cylinder in FIG. 1A that undergoes shape-shifting into spheres upon ultrasonication, as shown in FIG. 1B;

FIG. 2: A-B show CLSM micrographs of triphasic PLGA micro-components. In FIG. 2A, the micro-components are cylinders having a pie-shaped orientation of the three respective phases. The cylinders are shape-shifted into spheres (where the three phases are of a stripe-type) upon ultrasonication, as shown in FIG. 2B. Importantly, the shape-shifted spheres retain their phase orientation and alignment upon ultrasonication;

FIG. 3: A-D show CLSM and SEM micrographs of biphasic PLGA/poly(methyl methacrylate) (PMMA) cylinders. The cylinders have a 20 μm length in FIG. 3A and a 50 μm length in FIG. 3C. FIG. 3B shows the shape-shifting of the 20 μm cylinders from FIG. 3A upon ultrasonication. FIG. 3D likewise shows the shape-shifting of the 50 μm cylinders from FIG. 1C upon ultrasonication;

FIG. 4: A-D show CLSM and SEM images of multiphasic micro-components in the shape of micro-cylinders, where one part/phase is pure PLGA and the other part/phase is mixture of PLGA and poly(vinyl cinnamate) (PVCi) at a 1:1 ratio. The cylinders have a 20 μm length in FIG. 4A and a 50 μm length in FIG. 4C. FIG. 4B shows the shape-shifting of the 20 μm cylinders from FIG. 4A upon ultrasonication. FIG. 4D likewise shows the shape-shifting of the 50 μm cylinders from FIG. 4C upon ultrasonication. PVCi is photocrosslinked before applying ultrasonication, and thus the PVCi phase/part maintains its morphology during the shape-shifting process. After shape-shifting, fragments are responsible for PVCi observed on the PLGA sphere (arrows in B and D); and

FIG. 5: A-E show another embodiment of shape-shifting in accordance with the present disclosure. FIG. 5A shows a cross-sectional CLSM image of a biphasic micro-cylinder composed of PVCi (blue phase) and PEO (green phase). FIG. 5B is an SEM image of a sectioned biphasic fiber with 20 μm in length. FIG. 5C is a CLSM image of a plane view of a biphasic micro-cylinder after photocrosslinking (Scale bar is 50 μm). FIG. 5D shows a shape-shifted micro-cylinder created by introducing dioxane as a stimulator molecule. Because photocrosslinked PVCi swells to a relatively large volume in the presence of dioxane, as compared to PEO, the micro-cylinder is bent toward PEO phase. As shown in CLSM, the PVCi phase (Blue part) is enlarged when the dioxane is introduced as an external stimulus. FIG. 5E depicts an optical microscopy image of a 20 μm micro-cylinder showing reversible switching. When the dioxane is removed (via drying), the micro-cylinder returns to its original shape and therefore the physical deformation/shape-shifting is reversible.

FIG. 6 is a schematic of an exemplary electrohydrodynamic (EHD) co-jetting apparatus used to fabricate multiphasic components, including multiphasic fibers and multiphasic particles.

FIG. 7 shows a schematic of another exemplary electrohydrodynamic (EHD) co-jetting apparatus used to fabricate multiphasic components, including a post-formation microsectioning process used to prepare bicompartmentalized microcylinders with pre-selected aspect ratios.

FIG. 8: A-C. FIG. 8A shows a schematic of an overview of certain aspects of the shape shifting capabilities of multiphasic components of the present inventive technology, including a graph showing temperature versus time and the impact of changing a glass transition temperature (Tg) of a polymeric material forming a multiphasic microcomponent. FIGS. 8B and 8C are SEM images of sectioned biphasic components, with a 50 μm length show an inset. In FIG. 8B, the biphasic particles having a microcylinder shape (e.g., while in a hydrophobic medium), while the particles transition to a spherical particle shape (e.g., while in a hydrophilic medium).

FIG. 9: A-C. FIG. 9A shows a schematic where a plurality of biphasic anisotropic particles is receptive to an external stimulus, in this embodiment ultrasound energy, that induces shape shifting from a microcylinder shape to a microspherical shape. FIG. 9B shows CLSM and SEM micrographs of the anisotropic particles having a cylindrical shape, while FIG. 9C shows CLSM and SEM images after ultrasonic treatment of the plurality of particles that induces formation of spheres.

FIG. 10: A-D. FIG. 10A shows CLSM and SEM micrographs of a plurality of biphasic anisotropic particles receptive to an external stimulus. The initial biphasic particle has a shape of a microcylinder with an average diameter of about 15 μm and an average length of about 30 μm, as shown in FIG. 10A. After application of an external stimulus, these particles are shown in FIG. 10B to shift to a spherical shape. Similarly, an alternative embodiment is shown in the CLSM and SEM images in FIG. 10C, where an initial biphasic particle similarly has a shape of a microcylinder, but has an average diameter of about 25 μm and an average length of about 70 μm. FIG. 10D shows CLSM and SEM images after treatment of the plurality of such particles with an external stimulus that induces a spherical shape.

FIG. 11: A-D. FIGS. 11A-11D shows a variety of distinct embodiments of multiphasic anisotropic particles receptive to an external stimulus demonstrating that the shape shifting process can be precisely defined by adjusting preselected starting materials. Each of FIGS. 11A-11D show a schematic of a plurality of microcylinders having distinct phase orientations with CLSM images of a first microcylinder shape (along with phase locations in the first shape) before application of an external stimuli compared to a second spherical shape after application of the external stimuli. The consistency of compartmentalization or phase orientations can be clearly observed even after shape-shifting.

FIG. 12 is a table summarizing needle arrangements for an electrohydrodynamic (EHD) co-jetting system and preparation procedures for forming a variety of different embodiments of multicompartmental multiphasic anisotropic particles having an initial microcylinder shape.

FIG. 13: A-E illustrate a schematic for preparing a plurality of multiphasic anisotropic particles receptive to an external stimulus in accordance with certain aspects of the present technology. FIG. 13A shows an electrohydrodynamic (EHD) co-jetting system for continuously forming multiphasic fibers, where well-aligned fibers are collected on a rotating wheel collector. FIG. 13B shows a bundle of the multiphasic fibers formed by the process of FIG. 13A. FIG. 13C shows subsequent micro-sectioning of the fiber bundles with a cryogenic process to form a plurality of microcylinder particles having preselected dimensions. After sectioning, large populations of micro-cylinders with substantially the same diameters and having well-defined and controllable lengths are obtained, as shown in the SEM image of typical microcylinders (sectioned at a length of 30 μm, scale bar: 100 μm) in FIG. 13D. Moreover, microcylinders with multiple, different compartments are prepared using a range of different needle sets in the electrohydrodynamic jetting process of FIG. 13A (see insets in FIG. 13A, including core/shell and dual-core/shell needle arrangements). FIG. 13E is an overview of shape reconfiguration techniques based on different embodiments of multicompartmental multiphasic anisotropic microcylinders according to certain aspects of the present technology, specifically: (i) shape-shifting, (ii) reversible switching, and (iii) three-way toggling.

FIG. 14: A-H show isotropic shape-shifting of microcylinders into microspheres in accordance with certain aspects of the present disclosure. FIG. 14A shows a schematic representation of the shape-shifting of bicompartmental/biphasic particles by ultrasound. FIGS. 14A-14D are corresponding CLSM and SEM (inset) images of bicompartmental microcylinders (30 μm in length) before and after shape-shifting (left to right, C depicts an intermediate shifted state). FIGS. 14E-14F shows reconfiguration of bicompartmental microcylinders (70 μm in length) before (left) and after (right) shape-shifting. FIGS. 14G-14H shows reconfiguration of tricompartmental/triphasic microcylinders before (left) and after (right) shape-shifting (scale bar: 20 μm).

FIG. 15: A-H show anisotropic shape-shifting of bicompartmental microcylinders. FIG. 15A is a schematic illustration of the shape-shifting from a microcylinder to an anisotropic shape. FIGS. 15B-15D show CLSM and SEM (inset) images of bicompartmental PMMA/PLGA microcylinders before (15B), in the midst of (15C) and after (15D) shape-shifting by exposure to an external stimulus, here an ultrasound treatment. Blue and red fluorescent dyes are incorporated in PLGA and PMMA, respectively. Upon ultrasound treatment, only the PLGA phases/compartments are reconfigured into spherical shapes (15C indicates an intermediate state). Scale bars in CLSM and SEM images are 20 μm and 10 μm, respectively. FIGS. 15E-15H are SEM images of various shapes of multicompartmental particles with different polymers that are produced from a similar process. From left to right: Bicompartmenal PLGA/(PLGA+PMMA)(15E), PVCi/PLGA microparticles (15F), tricompartmental PLGA/(PLGA+PVCi)/PLGA microparticles (15G), bicompartmental PS/PLGA microparticles (15H). All scale bars are 10 μm.

FIG. 16: A-F show two-way reversible shape-switching of bicompartmental microcylinders in accordance with certain variations of the present technology. FIG. 16A is a schematic illustration of two-way shape-switching of hydrogel/PLGA core/shell microcylinders by swelling-deswelling of a core compartment upon exposure to a change in an environmental stimuli (here from water to dry state). FIG. 16B is an SEM and fluorescence OM (inset) images of Hydrogel/PLGA microcylinders with a length of 50 μm (scale bar: 25 μm). OM images are obtained in the presence of water, showing the core compartment is selectively swollen by water. FIG. 16C are OM images of the swelling and deswelling actions of the core compartments that expand reversibly in water (scale bar: 50 μm). FIG. 16D is a schematic illustration of the reversible bending caused by the swelling-deswelling of one compartment only. FIG. 16E are SEM and CLSM (inset) images of an embodiment of multiphasic/multicompartmental PVCi/PEO microcylinders (scale bar: 10 μm). FIG. 16F shows OM images of selective swelling cycles of PVCi in the presence of dioxane, resulting in reversible actuation (scale bar: 50 μm).

FIG. 17: A-E shows a plurality of multiphasic microparticles receptive to an external stimulus in accordance with certain aspects of the present technology which exhibit three-way shape toggling. FIG. 17A is a schematic diagram of microcylinders that are comprised of compartments comprising a hydrogel and an organogel. FIG. 17B shows OM images of bicompartmental PVCi/Hydrogel microcylinders (sectioned at a length of 50 μm) showing the full range of actuation, as a solvent environment changes from 100 water to 100% dioxane (left: in water, center: dry state, and right: in dioxane; scale bar: 50 μm). FIG. 17C shows OM images (left) and fluorescence OM (right) images of 200 μm microcylinders showing shape-toggling behavior (scale bar: 100 μm). FIG. 17D shows actuation behavior over time for longer multiphasic fibers undergoing shifting. FIG. 17E are OM images of the actuation angles controlled by adjusting the ratios of dioxane and water. The ratios indicate dioxane and water, respectively (scale bar: 50 μm).

FIG. 18: A-E. FIG. 18A shows a schematic outlining reconfiguration/shape-shifting of polymeric microcylinders into microspheres in accordance with certain aspects of the present disclosure. FIG. 18A is a graphical representation showing a relationship for temperature versus time for an exemplary shape-shifting process. When shape-shifting polymeric microcylinders that are responsive to an external stimulus like ultrasound are treated with ultrasonication, an increase in temperature above the glass transition temperature (T_{g}) of the polymer causes cylindrical particles (18B) to take on a spherical envelope (18C). As control experiments, the microcylinders are treated with ultrasound in an ice bath (18D) for keeping the temperature below T_{g} of polymer during sonication, and in heptane (bₚ=98° C.) (18E), an apolar medium, which does not have the driving force to minimize the surface area of the particles. In both cases, no change in particle shapes is observed.

FIG. 19: A-D show CLSM images demonstrating shape-shifting of multicompartmental microcylinders comprising poly(lactide-co-glycolide) (PLGA) prepared in accordance with certain aspects of the present technology. Upon ultrasound treatment, “pie-shaped” tricompartmental particles are formed (19A) and hepta-compartmental (19C) microcylinders are reconfigured to microspheres, FIGS. 19B and 19D, respectively. CLSM and OM images show the heptacompartmental particles with one compartment having a green colorant (PTDPV) and the others having a blue colorant (MEHPV) are successfully confined in one compartment through the shape-shifting process.

FIG. 20: A-C show examples of anisotropic shape-shifting of bicompartmental microcylinders before and after exposure to an external stimulus to which they are responsive (ultrasound treatment). CLSM images of the particles from 20A-20C correspond to the respective SEM images of FIGS. 15E-15G. Scale bars are all 20 μm.

FIG. 21 shows comparative information for experimentally obtained shape-shifted particles with expected equilibrium envelopes in accordance with certain aspects of the inventive technology. CLSM images and the corresponding models show bicompartmental shape-shifting particles comprising a first phase or compartment comprising PLGA and a second phase or compartment comprising PLGA (21A-21C) that shift from microcylinders to microspheres, while alternative bicompartmental shape-shifting particles are shown in 21D-21F that comprise a first phase or compartment comprising PVCi and a second phase or compartment comprising PLGA. The shifting behavior is FIGS. 21A-21C is isotropic, while the shifting behavior in FIGS. 21D-21F is anisotropic (as only one phase is responsive to an external stimulus).

FIG. 22 is representative of an exemplary chemical structure of a hydrogel comprising polyethylene glycol (PEG) diglycidyl ether and a branched PEI poly(ethyleneimine) (PEI) mixed at a 1:1 (w/w) ratio. The amine groups in PEI and epoxide groups in PEG can be crosslinked to form a hydrogel for use as a material in the multiphasic microparticles in accordance with certain variations of the present teachings;

FIG. 23 is representative of an exemplary chemical structure of a first chemical structure of a PVCi (before and after photocrosslinking) and a second chemical structure of polyethylene oxide (PEO) for use as materials in the multiphasic microparticles in accordance with certain variations of the present teachings.

FIG. 24: A-G show aligned fibers formed in accordance with certain variations of the present teachings comprising PVCi, PEO, and PLGA. More specifically, FIG. 24 shows certain embodiments of microfibers comprising distinct phases or compartments with an arrangement of dual core phases (comprising PVCi and PEO, respectively) and a shell phase surrounding the dual core (comprising PLGA). FIG. 24A is an SEM image of the aligned fibers comprising PVCi, PEO, and PLGA (scale Bar: 250 μm) formed by electrohydrodynamic jetting. FIG. 24B is an SEM image of a cross-sectional view of the bundle of fibers shown in FIG. 24A (scale bar: 25 μm). FIG. 24C is a three-dimensional (3D) CLSM image of the aligned fibers. Green and blue colorants (representing PEO and PVCi respectively) are indicated as 1 or 2. (There is no fluorescent dye in an external PLGA shell compartment (scale bar: 30 μm). FIGS. 24D-24G show cross-sectional views of microfibers of certain embodiments of the present technology comprising distinct phases or compartments of PVCi, PEO, and PLGA having dual core phases surrounded by a shell phase (scale bar: 50 μm), overlaid with CLSM and differential interference contrast (DIC) images. A bicompartmental dual-core is clearly observed in a PLGA shell phase, as reflected by FIG. 24G.

FIG. 25: A-B show CLSM and DIC images of certain embodiments of the present disclosure of microfibers comprising distinct phases or compartments with an arrangement of dual core phases (comprising PVCi and PEO, respectively) and a shell phase surrounding the dual core (comprising PLGA). FIG. 25A is a low magnification of a cross-sectional view (core flow rate: 0.02 ml/h). FIG. 25B shows cross-sectional views of the microfibers formed as a function of core flow rates varying from 0.02 to 0.05 ml/h (shell flow rate: 0.1 ml/h, scale bars: 50 μm).

FIG. 26: A-C characterizes certain embodiments of the present disclosure comprising microfibers having distinct phases or compartments with an arrangement of dual core phases (comprising PVCi and PEO, respectively) and a shell phase surrounding the dual core (comprising PLGA). FIG. 26A is an SEM image of as-prepared PVCi/PEO@PLGA microfibers (scale bar: 30 μm). FIG. 26B shows bicompartmental PVCi/PEO microfibers after photocrosslinking of PVCi and removing the PLGA shell (scale bar: 30 μm). FIG. 26C shows FTIR spectra of as-prepared PVCi/PEO@PLGA microfibers (black), after photocrosslinking (red) and after the removal of PLGA by dioxane (green).

FIG. 27 is an SEM image of a bicompartmental PVCi/PEO microcylinder formed in accordance with certain aspects of the present disclosure after dissolving PEO in DI water. Scale bar is 3 μm.

FIG. 28: A-C show OM images of bicompartmental PVCi/PEO microcylinders formed in accordance with certain embodiments of the present disclosure, 20 μm (FIG. 28A) 50 μm (FIG. 28B) and 100 μm (FIG. 28C) in length, showing reversible actuation. The actuation/shifting behavior is observed by sequential introduction of dioxane, and the actuation angle of each cylinder is measured (scale bars: A. 20 μm and B, C. 50 μm).

FIG. 29: A-B. FIG. 28 A shows actuation angles of microcylinders formed in accordance with certain embodiments of the present disclosure exposed to dioxane and dry states. The microcylinders have different cylinder lengths. FIG. 29B shows a linear relationship between actuation angle differences and cylinder lengths.

FIG. 30: A-B. FIG. 30A shows a chemical structure of PVCi and the hydrogel for use as materials in multiphasic microparticles in accordance with certain variations of the present teachings. FIG. 30B shows cross-sectional CLSM and DIC images of microfibers having a dual phase core configuration, wherein each compartment or phase comprises PVCi and hydrogel and the dual-core is surrounded by a shell phase comprising PLGA (“PVCi/Hydrogel@PLGA”).

FIG. 31: A-D. FIG. 31A shows an SEM image of as-prepared PVCi/Hydrogel@PLGA microfiber prepared in accordance with certain embodiments of the present disclosure (scale bar: 50 μm). FIG. 31B is an SEM image of PVCi/Hydrogel microfibers after photocrosslinking of PVCi, thermal crosslinking of the hydrogel and the shell (PLGA) dissolution (scale bar: 50 μm). FIG. 31C shows hydrogel microfibers after thermal crosslinking of the hydrogel compartment only (without photocrosslinking) and the PLGA dissolution (scale bar: 50 μm). An inset shows a cross-sectional view of hemisphere-cylinder morphology (scale bar: 2 μm). FIG. 31D shows PVCi/Hydrogel microcylinders having a length of 200 μm (scale bar: 20 μm).

FIG. 32 shows FTIR spectra of PVCi/hydrogel@PLGA microfibers (black), after photocrosslinking (red) and the PLGA removal (green). As a control experiment, FTIR spectrum of the hydrogel microfibers which is obtained by PLGA removal without photocrosslinking is also provided (blue).

FIG. 33 shows OM images of bicompartmental PVCi/Hydrogel microcylinder in a water environment and in an environment having a different pH level of 4 (scale bar 50 μm).

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as Well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various phases, elements, components, regions, layers and/or sections, these elements, components, regions, layers and/or sections should not be limited by these terms. These terms may be only used to distinguish one phase, element, component, region, layer or section from another region, layer or section. Terms such as “first,” “second,” and other numerical terms when used herein do not imply a sequence or order unless clearly indicated by the context. Thus, a first phase, element, component, region, layer or section discussed below could be termed a second phase, element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Throughout this disclosure, the numerical values represent approximate measures or limits to ranges to encompass minor deviations from the given values and embodiments having about the value mentioned as well as those having exactly the value mentioned. Other than in the working examples provides at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

The present disclosure provides novel polymeric anisotropic particles comprising at least two distinct phases that are capable of reconfiguration or “shape-shifting” by changing from an initial state (e.g., shape A) to an altered and distinct state (e.g., shape B) upon exposure to an external stimulus. Thus, in certain aspects, the multiphasic anisotropic micro-components of the present disclosure have at least one phase that is dynamic or changes its physical or chemical properties in response to a change in the surrounding physical, chemical, or biological environment. For instance, in certain variations, multiphasic anisotropic micro-components can be formed to have at least one phase that swells or has an altered shape when it is exposed to such an external stimulus. As such, the physical or chemical properties of the multiphasic micro-components can change and induce or enhance release of an ingredient within the micro-component, such as an active agent, like a drug, a bioactive material, a fragrance, a chemical, and the like. In certain aspects, the change or response observed in the phase may be at least partially reversible, once the stimulus is taken away or modified.

In certain variations, the multiphasic micro-component comprises at least one component that is responsive to an external stimulus; so that the micro-component exhibits a substantial physical deformation (e.g., transformation or reconfiguration) in response to the presence of, or alternately a change in, such an external stimulus. Exemplary non-limiting stimuli to which multiphasic micro-components can be designed to respond include temperature, light, pH, moisture and/or humidity, ionic strength, hydrophobicity/hydrophilicity, a controllable external field, like an energy field, an electric field, a magnetic field, or a sonic energy field, or various stimulator or activator chemicals stemming from either the human body or the environment, such as a solvent or a biomolecule. In certain variations, the external stimulus comprises ultrasound energy, which promotes heating of the multiphasic microcomponents.

Anisotropic multiphasic micro-components capable of shape-shifting are desirable for use in a variety of applications for micro/nanotechnology. In various aspects, multiphasic components suitable for use with the present technology, such as biphasic micro-components capable of shape-shifting, comprise one or more polymers. In certain aspects, one or more phases of the multiphasic micro-components include a component that is responsive to a controllable external stimulus. Such multiphasic micro-components can be made in a process that uses electrified jetting techniques to fabricate polymer-based shapes. In certain aspects, such multiphasic micro-components are nano-component particles. In other aspects, the multiphasic micro-components are micro-component particles.

A “nano-component” is a material that has a variety of shapes or morphologies, however, generally has at least one spatial dimension that is less than about 10 μm (i.e., 10,000 nm). The term “nano-sized” or “nanometer-sized” is generally understood by those of skill in the art to mean less than about 10 μm (i.e., 10,000 nm), optionally less than about 2 μm (i.e., less than about 2,000 nm), optionally less than about 0.5 μm (i.e., 500 nm), and in certain aspects, less than about 200 nm. In certain aspects, a nano-component as used herein has at least one spatial dimension that is greater than about 1 nm and less than about 10,000 nm. In certain aspects, a nano-component has at least one spatial dimension of about 5 to about 5,000 nm. In some aspects, at least one spatial dimension of the nano-component is about 20 to about 2,000 nm. In still other variations, nano-components have at least one spatial dimension of about 50 to about 500 nm.

A “micro-component” is a material that has a variety of shapes or morphologies, however, generally has at least one spatial dimension that is less than about 1,000 μm (1 mm), optionally less than 500 μm, optionally less than 250 μm, optionally less than 100 μm, optionally less than about 75 μm, optionally less than about 50 μm, optionally less than about 25 μm, optionally less than about 20 μm, optionally less than about 10 μm (i.e., 10,000 nm), optionally less than or equal to about 5 μm (i.e., 5,000 nm) and in certain aspects, optionally less than about 1 μm (i.e., 1,000 nm). Of course, as appreciated by those of skill in the art, other dimensions of the particle may be significantly greater than the dimension falling within the nano or micro range.

As mentioned above, the term micro-components is used interchangeably with the term “nano-objects,” “nano-components,” and “micro-objects”. Such micro-components may have a variety of geometries or morphologies, including, by way of non-limiting example, spheres, ovals, rectangles, polygons, disks, ellipsoids, toroids, cones, pyramids, rods/cylinders, beads-on-a-string, fibers, and the like. Micro-fibers generally have an elongated axial dimension that is substantially longer than the other dimensions of the micro-fiber. A “micro-component” generally refers to a micro-component where all three spatial dimensions are micro-sized and less than or equal to about 1 mm (e.g., less than about 1,000 μm). Again, in certain variations, nano-particles have at least one spatial dimension of about less than about 5,000 nm (about 5 μm). Micro-spheres and nano-spheres are substantially spherical. Micro-rods and nano-rods are components that are substantially cylindrical or rod-shaped.

In certain aspects, the multiphasic particles comprise materials in a solid phase or a semi-solid phase, although liquid phases are contemplated in certain variations. The term phase as used herein includes physically distinct compartments within a micro-component and the terms are used interchangeably herein. A structural component is intended to mean a compound of the multiphasic particle that renders it solid. In certain aspects, at least one phase of the multiphasic particle comprises at least one polymer. As appreciated by one of skill in the art, the first phase and the second phase (or additional distinct phases) can optionally include other polymers that are the same or different from one another. Further, one or more phases of the multiphasic micro-components optionally includes a component that is responsive to a controllable external stimulus. In certain variations, such a component responsive to an external stimulus is a polymer. Thus, in certain aspects, the multiphasic component comprises a first phase having a first polymer and a second distinct phase having a second polymer. Thus, one or more of the first polymer and second polymer is preferably the component that is responsive to the external stimulus. A multiphasic composition may include a variety of polymers, so that the multiphasic micro-component may comprise a first phase and a second distinct phase, where the first phase comprises the first polymer (or a plurality of polymers) and the second phase likewise optionally comprises the second polymer (or plurality of polymers). When present, one or more of the first polymers in the first phase is optionally distinct from the one or more second polymers present in the second phase. Stated in another way, the first phase may comprise at least one distinct polymer from the second phase. In certain embodiment, the at least one distinct polymer can be selected to be responsive to an external stimulus (optionally a different stimulus than the one to which the first polymer is responsive) or may be relatively inert in the presence of the external stimulus.

Multiple phases of the composition may each respectively comprise a plurality of distinct polymers. In other aspects, one or more of the distinct phases of the multiphasic particle may have a common polymer. Optionally, this common polymer may be the component responsive to the external stimulus. The first and second phases (or additional phases) may contain one or more of the same polymers or different polymer mixtures. In certain aspects, the multiphasic particles comprise multiple polymers. Thus, in various aspects, the multiphasic components suitable for use in the present teachings include a first phase and at least one additional phase that is distinct from the first phase. In certain preferred embodiments, the multiphasic particles are anisotropic. In certain preferred embodiments, at least one of the first polymer or the second polymer is selected to be the component that is responsive to the external stimulus. In other aspects, the multiphasic components optionally include multiple distinct phases, for example three or more distinct phases.

As used herein, “multiphase” or “multiphasic” means that at least two phases or “compartments” occupy separate, but distinct physical spaces to form the particle shape defining distinct “compartments.” In certain embodiments, such phases are in direct contact with one another (e.g., they are not separated by a barrier and they are not emulsified or mixed to any significant degree). By the term “phase” it is meant that a portion, domain, or region of a component is chemically and/or physically distinct from another portion, domain, or region of the component, for example a phase may have one average composition distinct from another phase having a different average composition. Each respective phase optionally occupies a spatially discrete region or compartment of the particle. In certain aspects, each respective phase of the multiphasic component can be exposed to an external environment (either in an initial state, an altered state, or in both initial and altered states), thus providing exposure of the respective phase surfaces of the multiphasic component to an external environment. The exposure of each respective surface of each phase provides enhanced environmental interface with the external stimulus/stimuli.

In various aspects of the present technology, the multiphasic micro-components are capable of so-called shape-shifting, which will be discussed herein. It should be noted that the term “shape-shifting” is not intended to be limited to a change in shape alone, but also includes other physical changes, such as a change in volume or other physical state. Generally, the multiphasic micro-components have an initial physical state, for example, an initial volume prior to exposure to an external stimulus or prior to a change in the level of such an external stimulus. In certain preferred embodiments, at least one of the first polymer or the second polymer is selected to be a component that is responsive to the external stimulus. Therefore, in certain variations, the volume of at least one phase of the multiphasic micro-components increases in the presence of one or more stimuli (or when one or more stimuli are changed), so that the initial state of the micro-component begins to deform (e.g., to expand).

It should be noted that while this example describes expansion or increased volume of one or more phases of the multiphasic micro-components when exposed to an external stimulus, some suitable materials may behave differently in the presence of such stimuli and the disclosure is not limited solely to expansion, but also encompasses contraction, as well as other detectable changes in shape. In accordance with the principles of the present disclosure, a multiphasic micro-component can undergo reversible deformation from the first physical state (e.g., occupying a first volume) to a second physical state (e.g., occupying a second volume) by having one or more of its phases change from a first physical state or volume to a second physical state or volume.

In accordance with certain aspects of the present disclosure, when the external stimulus is removed or returns to its initial level, then the component responsive to the external stimulus contained in one or more phases (e.g., one or more responsive polymers) reverts back to its original physical state due to elastic deformation. Elastic deformation generally refers to deformation that is nonpermanent and is recovered upon release of an applied stress or stimulus. By inelastic deformation, it is meant that the polymer or material undergoes substantially permanent, non-recoverable plastic deformation with the application of an applied stress above a specific threshold, for example, above a yield strength (σ_{y}) of the material. Thus, the deformation of a polymer or other material can transform from elastic to plastic under applied stress. In other words, in certain embodiments, depending on the selection of a polymer or material for a multiphasic micro-component, the micro-component has a first state and after absorption or interaction with the external stimulus (e.g., an applied stress), the polymer or material can be substantially deformed in an irreversible manner to a second state, and thus is incapable of reverting back to its original, first state. In other embodiments, the multiphasic micro-components polymer material is capable of substantially reversible deformation, meaning that the material undergoes reversible elastic deformation and can recover to its initial physical state after the stimulus is modified or removed.

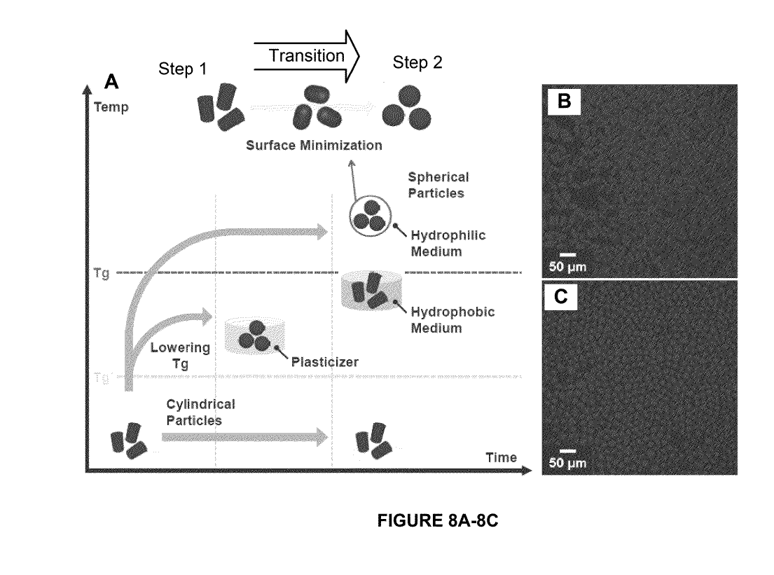

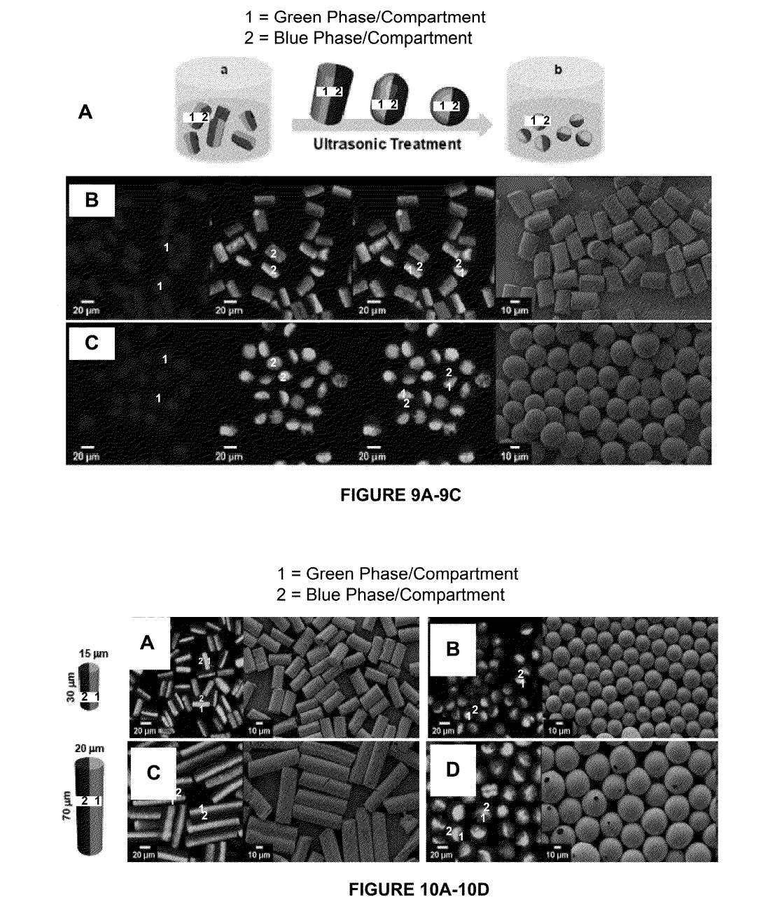

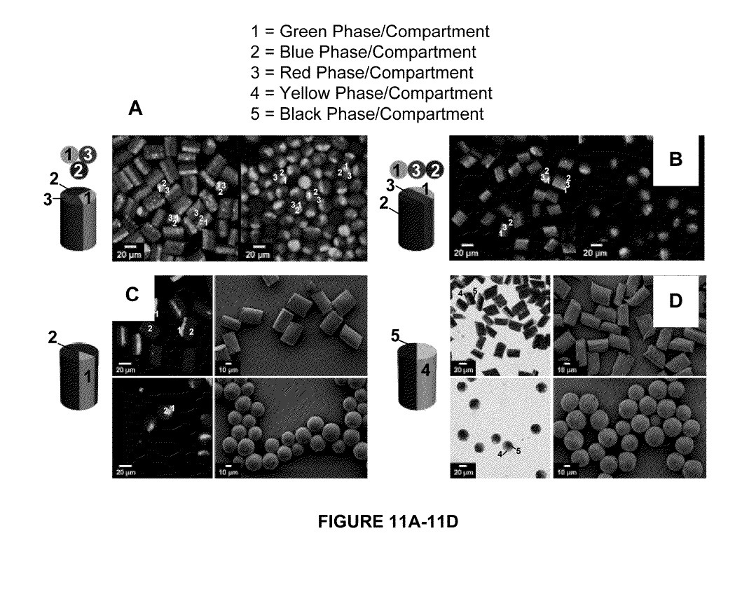

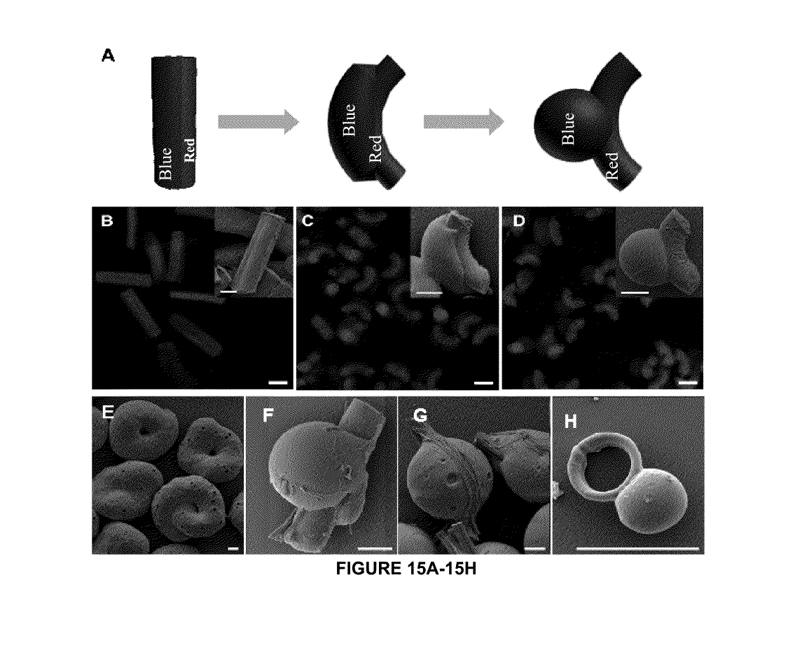

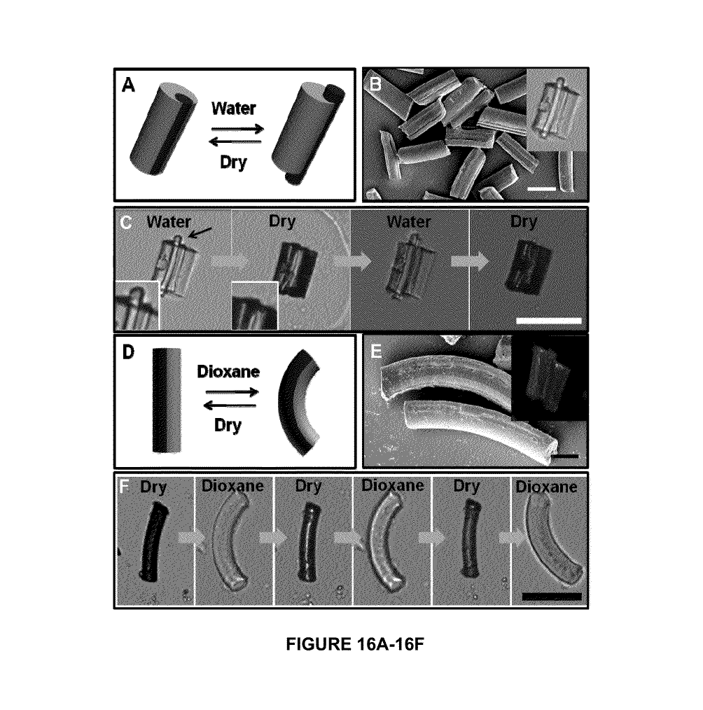

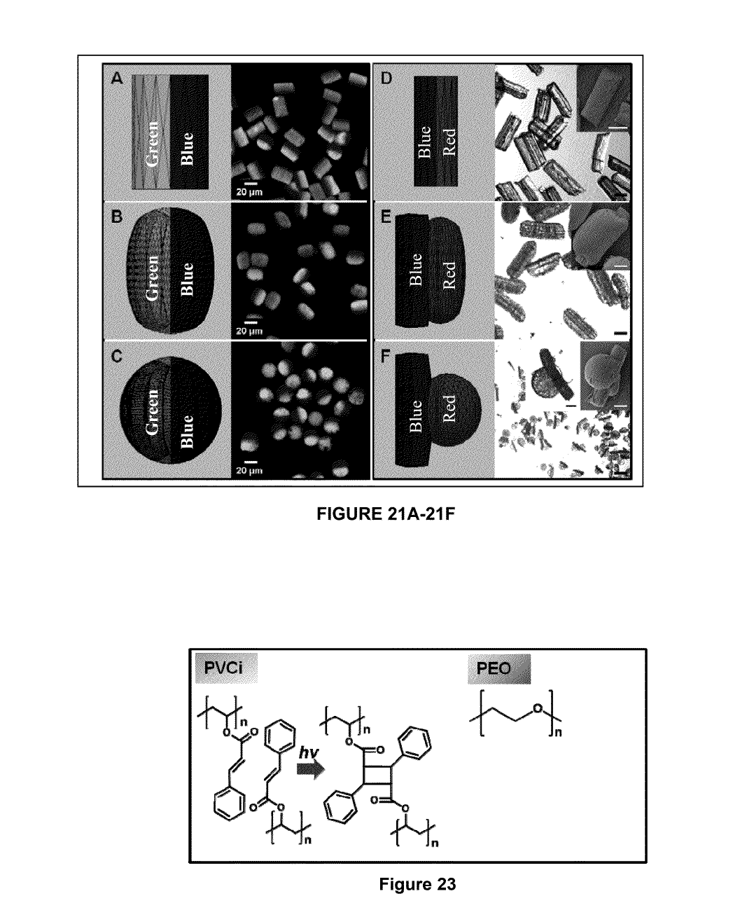

In certain variations of the present teachings, the reconfiguration or shape shifting is of an “anisotropic” nature, while in other alternative variations the reconfiguration or shape shifting may be of an “isotropic” nature. By isotropic shifting, it is meant that a physical transformation induced or promoted by an external stimulus occurs to multiple phases of the multiphasic microcomponent, so that the multiple phases are affected in a similar way and thus transformation is isotropic. See for example, FIGS. 21A-21C and also, FIGS. 8-9, 14, and 18. In anisotropic transformation, only certain select phases are affected by an external stimulus (or alternatively distinct phases may react in opposite manners), such that the transformation is uneven or anisotropic. See for example, FIGS. 21D-21F and also, FIGS. 15-16.

By “substantial” or “substantially” it is meant that the materials in one or more phases of the micro-component exhibit the stated property or undergo the stated action to the extent that the desired effect or result is achieved. For example, where a polymer or other material is substantially deformed, it undergoes a physical deformation that is detectable. In certain aspects, “substantial deformation” refers to a quantifiable change in shape, size, and/or orientation of phases within the micro-component. When a material undergoes substantially irreversible deformation, the deformation after exposure to the external stimuli leads to a desired effect of shape-shifting of the micro-component (such as a change in shape or volume), which is on the whole permanent, irreversible plastic deformation, even though some of the material may experience some elastic deformation. Likewise, when a material undergoes substantially reversible deformation, the deformation after exposure to the external stimuli leads to a desired effect of shape-shifting of the micro-component (such as a change in shape or volume) is elastic deformation, so that on the whole the material can revert back to its original state, even though some of the material may experience some irreversible plastic deformation.

Many polymeric materials have elastic and/or resilient properties (also referred to as “polymer memory recall”) and are capable of elastic deformation, where such materials reversibly expand to a larger volume by interaction with some external stimulus and then return to an original contracted state post-deformation. In other words, such materials, responsive to an external stimulus, spring back or recover (e.g., contract) to an original initial state after removing the source of the physical stress or other external stimulus. In certain variations, such responsive materials are suitable for use with the present teachings and will be described in greater detail below.

In this manner, the present disclosure provides methods of controlling the shape of a micro-component. The shape-shifting in the multiphasic micro-component is influenced by a controllable, external stimulus. Thus, the methods of the present disclosure optionally comprise exposing a multiphasic micro-component, capable of substantial deformation, to an external stimulus. Such exposure may include both introducing the external stimulus in the presence of the multiphasic micro-component, where it was previously absent, as well as changing the level (e.g., quantity or quality) of an external stimulus to induce the shape-shifting behavior. In certain variations, the external stimulus is selected from the group consisting of: temperature, pressure, light, pH, ionic strength, hydrophobicity/hydrophilicity, solvent, concentration, humidity, moisture, a stimulator chemical (meaning a chemical or molecule that stimulates the desired physical change in the multiphasic micro-component), sonic energy, electric energy, pressure, magnetic fields, and combinations thereof. It should be noted that these external stimuli are preferred; however, the external stimulus is not limited to solely these specific stimuli, but rather may include a variety of stimuli known or to be discovered in the art. As discussed above, the multiphasic micro-component preferably comprises a first phase and at least one additional phase distinct from the first phase, where at least one of the first phase and at least one additional phase comprises a polymer. In certain variations, the first phase comprises a first polymer and at least one additional phase distinct from the first phase comprises a second distinct polymer. The first polymer, and optionally the second polymer, may be the component in multiphasic micro-component that is responsive to an external stimulus. In certain embodiments, one or more of the first phase and the at least one additional phase exhibits the substantial deformation resulting in (i) a change in shape, (ii) a change in volume, or (iii) a change in both shape and volume of at least one of the first phase and the at least one additional phase when the multiphasic micro-component is subjected to certain methods of the present teachings.

In certain variations, the exposing further comprises changing the external stimulus from a first level to a second distinct level. By way of example, changing temperature, pH, pressure, light levels, ionic strength, hydrophobicity/hydrophilicity, concentration of particular chemicals or molecules, moisture, humidity, sonic energy, electric energy, pressure, magnetic fields, and combinations thereof, can achieve such a change in the external stimulus during the exposing step. In other variations, the exposing may further comprise introducing the external stimulus to the multiphasic micro-component, where it was previously absent. For example, the multiphasic micro-component may be exposed to newly introduced solvent, changes in solvent, humidity, moisture, hydrophobicity/hydrophilicity, or a stimulator chemical (meaning a chemical or molecule that stimulates or induces the desired physical change in the multiphasic micro-component), sonic energy, electric energy, pressure, magnetic fields, and combinations thereof. In certain preferred variations, the preferred external stimulus is a change of temperature, a change of pH, a change of ionic strength, hydrophobicity/hydrophilicity, presence of a biomolecule as a stimulator, or application of a sonication to the micro-components.

In yet other variations, the methods optionally comprise removing or altering the application of the external stimulus so that during the exposing of the external stimulus, the multiphasic micro-component is deformed from a first state to a second distinct state. After removing or changing the external stimulus, the multiphasic micro-component substantially returns to the first state by substantially reversible deformation, in accordance with the discussion above.

In accordance with various embodiments of the present disclosure, a multiphasic micro-component capable of shape-shifting is provided that has a first phase and at least one additional phase distinct from the first phase comprising a polymer. In certain preferred variations, the multiphasic micro-component capable of shape-shifting has a first phase comprising a first polymer and at least one additional phase distinct from the first phase comprising a second polymer. One or more of the first phase and the additional phase(s) exhibits a substantial physical deformation in response to the presence of or a change in an external stimulus. Thus, the shape-shifting process can be precisely controlled by selection of the starting materials in accordance with the present disclosure.

In certain aspects, multiphasic micro-components capable of shape-shifting can be formed by electrified jetting of materials that comprise one or more polymers, such as that disclosed by Roh et al. in “Biphasic Janus Particles With Nanoscale Anisotropy”, Nature Materials, Vol. 4, pp. 759-763 (October, 2005), as well as in U.S. Pat. No. 7,767,017, issued on Aug. 3, 2010 entitled “Multiphasic Nanoparticles”; U.S. Publication No. 2007/0237800 (U.S. application Ser. No. 11/763,842) entitled “Multi-Phasic Biofunctional Nano-Components And Methods for Use Thereof”; U.S. Publication No. 2010-0038830 (U.S. application Ser. No. 12/257,945) entitled “Method For Forming Biodegradable Nano-Components With Controlled Shapes And Sizes Via Electrified Jetting”; all of which are to Lahann et al. and assigned to a common assignee as the present disclosure. The contents of each of these respective patent references are hereby incorporated by reference in their respective entireties. Any other references discussed in this application are likewise expressly incorporated by reference in their respective entireties.

Hence, in various aspects, multiphasic micro-components and nano-components capable of shape-shifting are made in accordance with a process involving electrified jetting used to create such anisotropic multiphasic micro-components. In suitable electrified jetting techniques, liquid jets having a nanometer- or micro-sized diameter are shaped using electro-hydrodynamic forces. When a pendant droplet of conductive liquid is exposed to an electric potential, for example, of a few kilovolts, the force balance between electric field and surface tension causes the meniscus of the pendent droplet to develop a conical shape, the so-called “Taylor cone.” Above a critical point, a highly charged liquid jet is ejected from the apex of the cone, thus forming a multiphasic micro-component, such as a particle or fiber. Such electrical jetting techniques can be used in accordance with the present teachings to fabricate anisotropic multiphasic micro-components/nano-components that can be useful for a wide variety of shape-shifting applications.

Multiphasic micro-components can be made of a wide variety of materials, including inorganic and organic materials. In various embodiments, at least one phase of the multiphasic micro-components comprises at least one polymer, copolymer, or polymer precursor (e.g., monomer(s)), referred to herein generally as a “polymer.” In certain aspects, multiple phases of the multiphasic micro-components each comprise one or more polymers. In various aspects, the particles are formed by jetting liquid streams comprising a material optionally selected from liquid solutions, curable polymer precursors or monomers, polymer solutions, and polymer melts. Thus, each respective phase of the final micro-component product is formed from a material originating in the respective liquid streams. Specifically, each phase optionally contains polymers or polymer precursors (which upon curing form polymers), such as biodegradable or non-biodegradable polymers, biocompatible polymers, or natural polymers can be used. The particles can be further treated, for example by subsequent cross-linking induced by heat or actinic radiation (e.g., photochemically induced). Moreover, the cross-linking may also immobilize active materials in the final product.

In various aspects, the use of the electric jetting methods provides greater control over the morphology and design of the micro-components as opposed to other methods of forming micro-components (such as sonication during liquid jetting and the like). For example, the liquid jetting in the presence of an electric field of the present disclosure permits the use of immiscible materials as the first and second phases, as well as miscible materials. The broad use of such materials is possible due to the rapidity of formation of particles and shapes when an electric field is applied. For many conventional methods of formation, the respective phases require immiscibility between the phases, however that is not a requirement with the electric jetting methods employed here. Further, the methods of forming the multiphasic micro-components by use of side-by-side electric jetting further provides a high degree of control over the ability to create a wide variety of shapes, including fibers and the like.

Morphological control can be achieved with the exemplary electric jetting formation methods described briefly herein and in more detail in U.S. Publication No. 2010/0038830. A composite liquid stream is ejected from the pendant cone, which can be fragmented to small droplets or sustained and elongated in the form of a continuous fiber. The size of the droplet and diameter of the fibrous jet can also be controlled. Such control is attained by changing either the material properties of jetting liquids or the working parameters of electrified jetting that breaks-up the jet stream. It should be appreciated, however, that the final morphology of the liquid jet is not always the same as those of the solid products collected on a substrate on which the jetted product is received. The shape of final products can also be controlled by a sol-gel transition process or by subsequent processing after formation by electric jetting. When electric jetting is used to form multiphasic micro-components in the form of fibers (for example, by electrospinning), a sol-gel transition can be intrinsic to the process, since the jetting liquids are polymer solutions or polymer melts, and solvent evaporation or a temperature drop below the thermal transition temperature during the jetting acts as a sol-gel treatment step.

Since the electrified jetting methods are related to electrohydrodynamic processes, the properties of the jetting liquid and operating parameters are interrelated. Moreover, when the jetting liquids are not one-component systems (i.e., mixtures of two or more compounds), the jetting liquid is a solution having properties governed by several parameters of the solvent and solutes. It should be appreciated that liquid properties, solution parameters, and operating parameters are related, as recognized by those of skill in the art. Relevant material properties include viscosity, surface tension, volatility, thermal and electrical conductivity, dielectric permittivity, and density. Relevant solution properties include concentrations, molecular weight, solvent mixtures, surfactants, doping agent, and cross-linking agents. Finally, relevant operating parameters include flow rate of the liquid streams, electric potential, temperature, humidity, and ambient pressure. With regard to the operating parameters, the average size and size distributions of the droplets in electrospraying with cone-jet mode seem to be dependent on the flow rate (pumping rate of the jetting liquids). At a fixed flow rate, one or several relatively monodisperse classes of micro- and nano-component diameters are formed. At minimum flow rate, the modality of the distributions and diameter of the droplet itself also show their minima. When the flow rate is changed, the electric field can be adjusted by changing either distance or electric potential between the electrodes in order to sustain a stable cone-jet mode. Higher flow rates may be accompanied by a higher electrical field applied for mass balance of jetting liquids. When the diameter of droplets is larger than desired, solvent evaporation does not fully occur before the droplets reach the collecting substrate, so the resulting droplets may be wet and flat.

The electric jetting methods can control one or more of: concentration of the polymer in the liquid streams, flow rate of the liquid streams, humidity, temperature, electrode design, and configuration of electrodes during the jetting process, which provides a high selectivity of particles having substantially the same shape, size, and orientation of a first phase and/or at least one additional phase. For example, in certain aspects, concentration of polymer and flow rates of the liquid streams are two significant variables controlled to provide a plurality of nano-particles having substantially the same shape, size, or phase orientation. In other aspects, the electrode geometry and configuration during the electrospraying process is employed to control micro-component size, shape, selectivity, and distribution.

In certain aspects, the plurality of micro-components formed in accordance with the present methods, includes controlling certain parameters during jetting to provide nano-components having a predetermined shape selected from the group consisting of: spheres, ovals, rectangles, polygons, disks, toroids, ellipsoids, cones, pyramids, rods, cylinders, and fibers, as described in more detail in U.S. Publication No. 2010/0038830. In certain alternate aspects, the electrified jetting techniques can be employed to create a shell and core configuration of phases, as well.

After formation of the multiphasic micro-component, the polymers can also be further modified by chemical or physical methods after formation via electrified jetting, such as by cross-linking, heat treatment, photochemical treatment, and/or changes in the chemical or physical environment. The polymer modification can optionally occur in a select portion or region of one or more of the multiple phases, or such polymer modification can occur to different degrees, potentially resulting in different materials or materials responses, as appreciated by one of skill in the art. Such polymer modification and/or treatment provides the ability to control release kinetics of respective phases, when desired.

Specific polymers, such as biodegradable or non-biodegradable polymers, biocompatible polymers, or natural polymers are particularly suitable for use in the electrified hydrodynamic jetting techniques. In one aspect, the first phase of the multiphasic micro-component comprises a first polymer and the second phase comprises a second polymer that is distinct from the first polymer. In preferred embodiments, the first phase or the at least one additional phase comprises a component or material responsive to an external stimulus, for example, a material that exhibits substantial deformation upon exposure to or a change in an external stimulus. Such a responsive component may be a polymer. Thus, in certain aspects different polymers can be used in at least two phases of the multiphasic micro-component composition. In certain respects, different polymers used in the different phases of the micro-component permit the ability to control the shape-shifting abilities of the multiphasic micro-component, permitting a wide array of design choices. Further, different polymers may be used to provide different active ingredient release kinetics, which can be useful in designing release of an active ingredient from one or more of the phases of the micro-component into the environment.

In various aspects, suitable polymers for use in the multiphasic micro-components are polyester polymers selected from the group consisting of polylactides, polyglycolides, co-polymers, derivatives, and combinations thereof. As discussed above, at least one of the phases of the multiphasic micro-components of the present disclosure comprises a material responsive to an external stimulus. In certain embodiments, such a material is preferably a polymer. One example of a preferred polymer that is responsive to an external stimulus (undergoing substantial deformation), includes poly(lactide-co-glycolide) (PLGA). Another non-limiting example of such a preferred responsive polymer material is poly(vinyl cinnamate) (PVCi), which can be cross-linked. Thus, such polymers may be combined with other polymers in the multiphasic micro-component, as discussed herein.

In certain variations, other suitable materials for use in forming one or more phases or compartments of the multiphasic micro-components comprise a hydrogel or an organogel. Generally, a hydrogel is a colloidal gel comprising particles that are dispersed in water or an aqueous medium. An organogel is similar to a hydrogel, but rather than having a plurality of particles dispersed in water or an aqueous carrier, the particles are dispersed in an organic liquid. One hydrogel suitable for use as a material for a compartment of the micro-components comprises polyethylene glycol (PEG) diglycidyl ether and a branched PEI poly(ethyleneimine) (PEI), representative structures are shown in FIG. 22. In certain variations, the PEG and PEI can be mixed at a 1:1 (w/w) ratio. The amine groups in PEI and epoxide groups in PEG can be crosslinked to form the hydrogel.

An organogel suitable for use as a material for a phase of the micro-components can comprise cross-linked PVCi. FIG. 23 is representative of an exemplary chemical structure of a first chemical structure of a PVCi (before and after photocrosslinking) and a second chemical structure of polyethylene oxide (PEO) for forming organogels for use as materials in the multiphasic microparticles in accordance with certain variations of the present teachings.

In certain embodiments, multiphasic micro-components can comprise a hydrogel in a first phase and an organogel in a second phase of the micro-component. Such micro-components can create fully reversible three-way shape-toggling, such as in biphasic microcylinders (where one compartment was composed of a hydrogel and the other of an organogel).

In certain embodiments, the phases of the multiphasic micro-component may be selected to dissolve or disintegrate at different rates. In this regard, the dissolution rate of the respective phases impacts the release rate of any active ingredient from each phase, thus providing control over the release kinetics and concentration of active ingredient to be delivered to target regions with each respective phase of the micro-component. As referred to herein, “dissolve” refers to physical disintegration, erosion, disruption and/or dissolution of a material. The phases may dissolve or disintegrate at different rates or have different solubilities (e.g., aqueous solubility) that impact the rate of active ingredient release. In certain embodiments, each phase may comprise one or more materials that dissolve or erode upon exposure to a solvent comprising a high concentration of water, such as serum, blood, bodily fluids, or saliva. In some variations, a phase may disintegrate into small pieces or may disintegrate to collectively form a colloid or gel. In some aspects, a phase of the multiphasic micro-component comprises a polymer that is insoluble or has limited solubility in water, but is dispersible in water, so that the polymer breaks down or erodes into small fragments. In other aspects, a polymer used in a phase of the multiphasic micro-component is insoluble in water, but swellable. In variations where a polymer does not fully break down during use, the polymer can be a water-repellant polymer or an aqueous-stable hydrophilic polymer, for example, certain types of cellulose. In various aspects, each phase of the multiphasic micro-component optionally comprises a combination of polymer materials.

Suitable non-limiting polymers for use in the multiphasic compositions include poly(lactide-co-glycolide) polymer (PLGA), poly(vinyl cinnamate) (PVCi), sodium polystyrene sulfonate (PSS), polyethers, such as a polyethylene oxide (PEO), polyoxyethylene glycol or polyethylene glycol (PEG), poly(methyl methacrylate) (PMMA), polyethylene imine (PEI), a biodegradable polymer such as a polylactic acid, polycaprolactone, polyglycolic acid, and copolymers, derivatives, and mixtures thereof. Other polymers include those well known to those of skill in the art used in pharmaceutical, oral care, and personal care compositions, such as polyvinylpyrrolidone. Specifically, at least one phase can be designed to have one or more of the following properties based upon material selection: hydrophobic, positively-charged (cationic), negatively-charged (anionic), polyethylene glycol (PEG)-ylated, covered with a zwitterion, hydrophobic, superhydrophobic (for example having with water contact angles in excess of 150°), hydrophilic, superhydrophilic (for example, where the water contact angle is near or at 0°), olephobic/lipophobic, olephilic/lipophilic, and/or nanostructured, among others. In other aspects, one or more polymers or materials used within a phase may be functionalized to subsequently undergo reaction with various moieties or substances after formation of the multiphasic particle, to provide desired surface properties or to contain various moieties presented on the phase surface, as recognized by those of skill in the art.

Water-soluble and/or hydrophilic polymers, which are cosmetically and pharmaceutically acceptable, include cellulose ether polymers, including those selected from the group consisting of hydroxyl alkyl cellulose, including hydroxypropyl methyl cellulose (HPMC), hydroxypropyl cellulose (HPC), hydroxyethyl cellulose (HEC), methyl cellulose (MC), carboxymethyl cellulose (CMC), and mixtures thereof. Other polymers among those useful herein include polyvinylpyrrolidone, vinyl acetate, polyvinylpyrrolidone-vinyl acetate copolymers, polyvinyl alcohol (PVA), acrylates and polyacrylic acid (PAA), including polyacrylate polymer, vinylcaprolactam/sodium acrylate polymers, methacrylates, poly(methyl methacrylate) (PMMA), poly(acryl amide-co-acrylic acid) (PAAm-co-AA), vinyl acetate and crotonic acid copolymers, polyacrylamide, polyethylene phosphonate, polybutene phosphonate, polystyrene, polyvinylphosphonates, polyalkylenes, and carboxy vinyl polymer. The multiphasic compositions may comprise derivatives, copolymers, and further combinations of such polymers, as well.

Other polymers or water-soluble fillers among those useful herein include, without limitation, sodium alginate, carrageenan, xanthan gum, gum acacia, Arabic gum, guar gum, pullulan, agar, chitin, chitosan, pectin, karaya gum, locust bean gum, various polysaccharides; starches such as maltodextrin, amylose, corn starch, potato starch, rice starch, tapioca starch, pea starch, sweet potato starch, barley starch, wheat starch, modified starch (e.g., hydroxypropylated high amylose starch), dextrin, levan, elsinan and gluten; and proteins such as collagen, whey protein isolate, casein, milk protein, soy protein, keratin, and gelatin.

Further, non-limiting examples of water insoluble or hydrophobic polymers include cellulose acetate, cellulose nitrate, ethylene-vinyl acetate copolymers, vinyl acetate homopolymer, ethyl cellulose, butyl cellulose, isopropyl cellulose, shellac, hydrophobic silicone polymer (e.g., dimethylsilicone), polymethyl methacrylate (PMMA), cellulose acetate phthalate and natural or synthetic rubber; siloxanes, such as polydimethylsiloxane (PMDS), polymers insoluble in organic solvents, such as cellulose, polyethylene, polypropylene, polyesters, polyurethane and nylon, including copolymers, derivatives, and combinations thereof. The polymers may be crosslinked after formation by application of heat, actinic radiation or other methods of curing and treating polymers known to those of skill in the art.

In certain variations, a pharmaceutically and/or cosmetically acceptable polymer for the composition of the first phase or at least one additional phase (e.g., first or second polymers) is selected from the group consisting of: biodegradable polymers, water soluble polymers, water dispersible polymers, water insoluble polymers, and combinations and co-polymers thereof. In certain preferred variations, the first polymer of the first phase of the multiphasic micro-component and the second polymer of the at least one additional phase comprises a pharmaceutically and/or cosmetically acceptable polymer is selected from the group consisting of: Suitable non-limiting polymers for use in the multiphasic compositions include poly(lactide-co-glycolide) polymer (PLGA), poly(vinyl cinnamate) (PVCi), sodium polystyrene sulfonate (PSS), polyethers, such as a polyethylene oxide (PEO), polyoxyethylene glycol or polyethylene glycol (PEG), poly(methyl methacrylate) (PMMA), polyethylene imine (PEI), polylactic acid, polycaprolactone, polyglycolic acid, poly(lactide-co-glycolide polymer (PLGA), polyvinylpyrrolidone, hydroxyl alkyl cellulose, hydroxypropyl methyl cellulose (HPMC), hydroxypropyl cellulose (HPC), hydroxyethyl cellulose (HEC), methyl cellulose (MC), carboxymethyl cellulose (CMC), vinyl acetate, polyvinylpyrrolidone-vinyl acetate copolymers, polyvinyl alcohol (PVA), polyacrylates, polyacrylic acid (PAA), vinylcaprolactam/sodium acrylate polymers, methacrylates, poly(acryl amide-co-acrylic acid) (PAAm-co-AA), vinyl acetate, crotonic acid copolymers, polyacrylamide, polyethylene phosphonate, polybutene phosphonate, polystyrenes, polyvinylphosphonates, polyalkylenes, carboxy vinyl polymer, cellulose acetate, cellulose nitrate, ethylene-vinyl acetate copolymers, vinyl acetate homopolymers, ethyl cellulose, butyl cellulose, isopropyl cellulose, shellac, siloxanes, polydimethylsiloxane, cellulose acetate phthalate, natural or synthetic rubber; cellulose, polyethylene, polypropylene, polyesters, polyurethane, nylon, and copolymers, derivatives, and mixtures thereof. Particularly preferred polymers include poly(lactide-co-glycolide) (PLGA), poly(vinyl cinnamate) (PVCi), poly(methyl methacrylate) (PMMA), poly(ethylene)oxide (PEO), and combinations thereof.

In various aspects of the present disclosure, the polymers are present in a liquid phase prior to electrified jetting or spraying at about 0.1 to about 100% by weight (on a wet basis). While the relative concentrations of polymers in a phase can vary greatly depending on the polymer, application, and process parameters used for forming the micro-component, in certain aspects, the polymer is optionally present at about 2% to about 50% by weight; optionally from about 3% to 15% by weight of the phase.

In yet another embodiment of the disclosure, multiphasic micro-components with selective chemical modification are provided. The micro-components are formed from one or more liquid streams that include one or more reactive components that react with a structural component (i.e., a polymer) thereby rendering a resulting surface of the multiphasic micro-components chemically modified as compared to the surface when the one or more reactive components are absent. For example, during the formation of multiphasic micro-components, reactive functional groups are optionally incorporated by adding appropriate components in each respective jetting solution. After jetting, the surface of the micro-component will have different functional groups at each respective phase surface corresponding to the materials present in each respective jetting solution. In some variations, the different phases are detected by optical or electronic sensors, or by fluorescent or electron microscopy, for example.

In one aspect, the first phase of the multiphasic micro-component comprises a first polymer and the second phase comprises a second polymer that is distinct from the first polymer. Preferably, at least one of the first polymer or the second polymer is a component that is responsive to the selected external stimulus. Thus, in certain aspects different polymers can be used in at least two phases of the multiphasic micro-component composition. In certain respects, different polymers used in the different phases of the multi-phasic micro-component permit different surface properties or colorant or active ingredient release kinetics, which can be useful in designing release of the active ingredient into the environment. Further, otherwise incompatible ingredients, such as incompatible active ingredients can be stored simultaneously under stable conditions in near proximity to one another. Thus, in certain embodiments, the first phase comprises materials compatible with the first active ingredient and the second phase similarly has materials compatible with the second active ingredient. Thus, a lipophilic, hydrophobic, or charged active ingredient (e.g., cationic or anionic) can be included in one phase of the multi-phasic micro-component and a hydrophilic or oppositely charged colorant or active ingredient can be included in a second phase; however both the first and second colorants/active ingredients are stored in close proximity to one another and can be delivered simultaneously to a target substrate.

Moreover, in certain embodiments, each phase can comprise a different moiety (e.g., each phase can be tagged with a different targeting moiety or active agent) or can optionally have different surface properties. Specifically, at least one phase can be selected to be hydrophilic, hydrophobic, positively charged (cationic), negatively charged (anionic), surface active agent modified (e.g., PEG-ylated or covered with a zwitterion), superhydrophobic, superhydrophilic, olephobic, olephilic, and/or nanostructured, as described above. A multiphasic micro-component phase can be designed to have such properties by providing such materials within the material forming the phase, or may be provided by subsequent treating, reacting, or coating of the exposed phase surface after formation of the multiphasic micro-component to achieve such properties. Polymers within a selected phase can further be modified to interact and/or react with certain target moieties. For example, reactive groups on a polymer in a first phase may be cationic and the desired moiety for the surface is anionic and will be attracted to the surface of the first phase. In other embodiments, the functional groups on the polymer may participate in a reaction with a functional group present on the moiety, such that they react and are bonded to the surface of the phase. For example, if a first phase of the multiphasic micro-component has a polymer with a —CHO functional group at the surface and the moiety to be attached to the first phase has a —CH₂NH₂ functional group, such groups have an affinity to form a —C═N covalent bond, thus, the surface of the first phase has an affixed moiety presented at the surface.

In various aspects, one or more exposed phase surfaces comprises a moiety. In certain aspects, the moiety may be provided to interact with the surrounding environment (for example, to avoid multiphasic micro-component detection by an immune system, provide optical properties to the multiphasic micro-component, provide binding to a biological or non-biological target, such as a medical device). In some aspects, the moiety is a binding moiety that provides the ability for the multiphasic micro-component to bind with a target. In certain aspects, the target may be an immune system cell, protein, enzyme, or other circulating agent associated with the animal). The following provides exemplary and non-limiting examples of suitable binding moieties for use with the multiphasic micro-components of the disclosure. Proteins, such as heat shock protein HSP70 for dendritic cells and folic acid to target cancer cells. Polysaccharides or sugars, such as silyilic acid for targeting leucocytes, targeting toxins such as saporin, antibodies, including CD 2, CD 3, CD 28, T-cells, and other suitable antibodies are listed in a Table at http://www.researchd.com/rdicdabs/cdindex.htm (Jun. 14, 2007), incorporated by reference. Binding moieties include aptamers, which are small oligonucleotides that specifically bind to certain target molecules, for example, Aptamer O-7 which binds to osteoblasts; Aptamer A-10 which binds to prostate cancer cells; and Aptamer TTA1, which binds to breast cancer cells. Other exemplary binding moieties include peptides, such as CGLIIQKNEC (CLT1) and CNAGESSKNC (CLT 2) for binding to clots. Various peptides are well known in the art for binding to cells in the brain, kidneys, lungs, skin, pancreas, intestine, uterus, adrenal gland, and prostate, including those described in Pasqualini et al., “Searching for a molecular address in the brain,” Mol. Psychiatry. 1(6) (1996) pp. 421-2 and Rajotte, et al., “Molecular heterogeneity of the vascular endothelium revealed by in vivo phage display,” J Clin Invest. 102(2) (1998) pp. 430-7, for example. Other binding biological binding moieties known or to be developed in the art are contemplated by the present disclosure.

Other conventional materials can be used to form the materials of respective phases, including solvents, plasticizers, cross-linking agents, surface active agents, fillers, bulking, or viscosity modifying agents, pH modifiers, pH buffers, antioxidants, impurities, UV stabilizers, and where appropriate, flavoring, or fragrance substances.

At least one phase of the multiphasic micro-component optionally comprises an active ingredient. An active ingredient is a compound or composition that diagnoses, prevents, or treats a physiological or psychological disorder or condition, or can provide a cosmetic or aesthetic benefit. In certain aspects, an active ingredient agent is targeted to a particular target, such as organs, tissues, medical implants or devices, hair, skin, mouth, eyes, circulatory system, and the like. For example, in various aspects, the multiphasic micro-components having one or more active ingredients can be used in various pharmaceutical and/or cosmetic compositions. A “pharmaceutically and/or cosmetically acceptable composition” refers to a material or combination of materials that are used with mammals or other organisms having acceptable toxicological properties for beneficial use with such an animal. Pharmaceutically and/or cosmetically acceptable compositions include drug and therapeutic compositions, oral care compositions, nutritional compositions, personal care compositions, cosmetic compositions, diagnostic compositions, and the like. In certain aspects, the pharmaceutically and/or cosmetically acceptable composition includes medical devices and implants, or surface films or coatings for such devices. Thus, in various aspects, the multiphasic micro-components may be used in a wide variety of different types of compositions having a bio-functional or bio-active material and are not limited to the variations described herein. However, the present disclosure contemplates multiphasic micro-components comprising one or more active ingredients that provides a diagnostic, therapeutic, prophylactic, cosmetic, sensory, and/or aesthetic benefit to an organism, such as a mammal. In certain aspects, an active ingredient prevents or treats a disease, disorder, or condition of hard or soft tissue in an organism, such as a mammal.

The ensuing description of suitable active ingredients is merely exemplary and should not be considered as limiting as to the scope of active ingredients which can be introduced into the multiphasic micro-components according to the present disclosure, as all suitable active ingredients known to those of skill in the art for these various types of compositions are contemplated. Suitable active ingredients for use in such pharmaceutically and/or cosmetically acceptable compositions are well known to those of skill in the art and include, by way of example, pharmaceutical active ingredients found in the Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, each incorporated herein by reference. Each additional reference cited or described herein is hereby expressly incorporated by reference in its respective entirety. Certain suitable active ingredients, or pharmaceutically active ingredients or drugs, are known to those of skill in the art and include, but are not limited to, low-molecular weight molecules, quantum dots, natural and artificial macromolecules, such as proteins, sugars, peptides, DNA, RNA, and the like, polymers, dyes and colorants, inorganic ingredients including nanoparticles, nano-materials, and nano-crystals, fragrances, and mixtures thereof.

A variety of low molecular weight molecules can be employed, particularly those having a molecular weight of less than about 10,000, optionally less than about 1,000, and optionally less than about 500. Such molecules include therapeutic drugs, which by way of non-limiting example includes chemotherapeutic drugs, such as doxorubicin (molecular mass of about 543.5 g/mol); paclitaxel or Taxol™ (molecular mass of about 853.9 g/mol), cholesterol lowering drug, lovastatin (molecular mass of about 404.5 g/mol), NSAID analgesic ibuprofen (molecular mass of 206.3 g/mol). Quantum dots are optically active nanostructures, for example, cadmium tellurium (CdTe). Macromolecules include a wide range of compounds, generally including polymers and biomolecules having relatively large molecular weights. Such macromolecules can be naturally occurring or synthesized. Any variety of polymers well known to those of skill in the art can be employed if the polymers are smaller than the phase in which they are distributed. Amino acids, peptides (amino acids linked via peptide bonds); polypeptides (linear chains of peptides); and proteins (primary, secondary, and tertiary folded polypeptides) are all contemplated as active ingredients. Exemplary active ingredient proteins include heat shock protein 70 (HSP70) for dendritic cells and folic acid for cancer cells. Exemplary toxins for use as active ingredients include saporin and Botulinum toxins. Exemplary sugars include silyilic acid leucocytes and glucuronic acid, for example. Useful nano-particles and nano-crystals generally having a particles size of less than about 50 nm, optionally less than about 20 nm, and in some aspects, less than 10 nm. Useful non-limiting active ingredient nano-particles include magnesium oxide, and metal based nano-particles, comprising gold, silver, and the like. Suitable active ingredient nano-crystals include magnetite (Fe₃O₄).

In other variations, the active ingredient of the multiphasic micro-components of the disclosure may be used for diagnostic purposes, such as in various diagnostic medical imaging procedures (for example, radiographic imaging (x-ray), fluorescence spectroscopy, Forster/fluorescent resonance energy-transfer (FRET), computed tomography (CT scan), magnetic resonance imaging (MRI), positron emission tomography (PET), other nuclear imaging, and the like). Active ingredients for use with diagnostic imaging include contrast agents, such as barium sulfate for use with MRI, for example, or fluorescein isothiocyanate (FITC).

In other aspects, the active ingredient may provide a nutritional, cosmetic, aesthetic, or sensory benefit to the organism via the multiphasic micro-components. As described above, various active ingredients are well known to those of skill in the art and include those outlined in the International Cosmetic Ingredient Dictionary and Handbook, referenced above. Various suitable active agents or ingredients are known to those of skill in the art.

In certain aspects, multiphasic micro-components can be provided in pharmaceutical compositions. In certain pharmaceutical compositions, the active ingredient is provided in a suitable pharmaceutical excipient, as are well known in the art. Thus, administration of multiphasic micro-components in a pharmaceutical composition can be, for example, intravenous, topical, subcutaneous, transcutaneous, intramuscular, oral, intra-joint, perenteral, peritoneal, intranasal, by inhalation, or within or coating a medical device or implant. Pharmaceutical compositions are optionally provided in the form of solid, semi-solid, lyophilized powder, or liquid dosage forms, such as, for example, tablets, pills, capsules, powders, solutions, suspensions, emulsions, suppositories, retention enemas, creams, ointments, lotions, aerosols or the like, in unit dosage forms suitable for administration of precise dosages.

As discussed above, certain suitable active ingredients for pharmaceutical compositions or nutritional compositions, are known to those of skill in the art and include, but are not limited to, low-molecular weight molecules, quantum dots, natural macromolecules, such as proteins, sugars, peptides, DNA, RNA, and the like, artificial macromolecules, polymers, dyes and colorants, inorganic ingredients including nano-materials and nano-crystals, fragrances, and mixtures thereof. By way of non-limiting example, the active ingredient can be a therapeutic drug that operates locally or systemically (non-localized) and may treat, prevent, or diagnose a wide variety of conditions or ailments. Active ingredients may be used to treat or prevent a disease, such as an infectious disease (a bacterial, viral, or fungal infection) or a degenerative disease (Alzheimer's, amyotrophic lateral sclerosis (ALS)). For example, active ingredients may treat an auto-immune disorder (e.g., rheumatoid arthritis, systemic lupus erythematosus (SLE), inflammatory bowel disease (IBD)), allergies, asthma, osteoarthritis, osteoporosis, cancer, diabetes, arteriosclerosis and cardiovascular disease, stroke, seizures, psychological disorders, pain, acne, caries, gingivitis, periodontitis, an H₂ antagonist, and the like. Various suitable active ingredients are disclosed in Merck Index, An Encyclopedia of Chemicals, Drugs, and Biologicals, Thirteenth Edition (2001) by Merck Research Laboratories and the International Cosmetic Ingredient Dictionary and Handbook, Tenth Ed., 2004 by Cosmetic Toiletry and Fragrance Association, and U.S. Pat. Nos. 6,589,562, 6,825,161, 6,063,365, and 6,491, 902, all to Shefer et al.

In various aspects, a multi-phasic micro-component delivers an effective amount of the active ingredient to a target region within an organism. An “effective” amount of an active ingredient is an amount that has a detectable effect for its intended purpose and/or benefit. Preferably, the effective amount is sufficient to have the desired therapeutic, nutritional, cleansing, aesthetic, diagnostic, and/or prophylactic effect on the target region of an organism (e.g., a mammal) to whom and/or to which the composition comprising the multi-phasic micro-components is administered. The specific effective amount of the active ingredient, including appropriate dosages and concentrations, will vary with such factors as the composition in which the active ingredient is provided, the site of intended delivery, the route of administration, the particular condition or subject being treated, the nature of concurrent therapy (if any), the specific active used, the specific dosage form, and the carrier employed, all of which are well known to those of skill in the art.

In certain aspects, a safe and effective amount of an active ingredient in a phase of a multiphasic micro-component is about 0.0001 to about 95 weight % of the total weight of phase (on a dry basis); optionally about 0.01 to about 90 weight %. It should be noted that where the multi-phasic micro-component is distributed in a carrier or composition, that the overall concentration will be significantly less than in the multi-phasic micro-component particles. In certain aspects, the active ingredient is present in a phase on a multi-phasic micro-component at a concentration of about 0.001 to about 75% of the total phase. In other aspects, the active ingredient is present at from about 0.01 to about 20%; optionally of about 1% to about 20%; and optionally 5% to about 20%. However, as discussed above, the concentration of active ingredient is highly dependent on various factors well known to those of skill in the art, including required dosage for the target region, bioavailability of the active ingredient and the release kinetics of the phase in which the active ingredient is located, among others.

In some aspects, it may be desirable to avoid detection by the animal's immune system, for example, to prevent removal from the body by macrophages and the like. Thus, one or more phases of the multiphasic micro-component can employ various methods to prevent an animal's immune system from identifying and removing the multi-phasic micro-component prior to delivery to the target site where the active ingredient can be delivered. For example, in certain aspects, the moieties on the surface of at least one phase include a “cloaking agent” that prevents the animal's immune system from recognizing a foreign body. Examples of such moieties include modified carbohydrates, such as sialic acid, dextran, pullulan, or glycolipids, hyalluronic acid, chitosan, polyethylene glycols, and combinations thereof. Other examples of immune system cloaking agents known in the art or to be discovered are further contemplated.

Suitable, non-limiting examples of active ingredients that can be incorporated into multi-phasic micro-components of the invention include the following drugs: 5-Fluorouracil (5-FU): an anti-metabolite drug commonly used in cancer treatment. Typical dosing begins with intravenous treatment at 400 mg/m² (i.e., per square meter of calculated body surface area) over 15 minutes as a bolus, then an ambulatory pump delivers 2,400 mg/m² as a continuous infusion over 46 hours. Suitable chemotherapeutic drugs can be divided into the following classes: alkylating agents, anti-metabolites, anthracyclines, plant alkaloids, topoisomerase inhibitors, monoclonal antibodies, and other anti-tumor agents. In addition to the chemotherapeutic drugs described above, namely doxorubicin, paclitaxel, other suitable chemotherapy drugs include tyrosine kinase inhibitor imatinib mesylate (Gleevec® or Glivec®), cisplatin, carboplatin, oxaliplatin, mechloethamine, cyclophosphamide, chlorambucil, azathioprine, mercaptopurine, pyrimidine, vincristine, vinblastine, vinorelbine, vindesine, podophyllotoxin (L01 CB), etoposide, docetaxel, topoisomerase inhibitors (L01 CB and L01XX), irinotecan, topotecan, amsacrine, etoposide, etoposide phosphate, teniposide, dactinomycin, and monoclonal antibodies, such as trastuzumab (Herceptin™), cetuximab, bevacizumab and rituximab (Rituxan™), among others.

In this regard, multi-phasic micro-components incorporating such a drug can be designed to deliver equivalent dosages at the cancer cells, thus potentially minimizing the amount delivered generally to the patient and minimizing collateral damage to other tissues.

In certain aspects, the multi-phasic micro-component comprises lovastatin, a cholesterol lowering and heart disease active ingredient, which can be included in at least one phase of the multiphasic micro-component compositions. In another aspect, a suitable active ingredient included in at least one phase of the multi-phasic micro-component is Phenyloin, an anticonvulsant agent (marketed as Dilantin® in the USA and as Epanutin® in the UK by Pfizer, Inc). Antibiotics can be incorporated into one or more phases of the multi-phasic micro-components, such as vancomycin, which is frequently used to treat infections, including those due to methicillin resistant staph aureus (MRSA). At least one phase of a multi-phasic micro-component optionally includes Cyclosporin, a lipophilic drug that is an immunosuppressant agent, widely used post-allogeneic organ transplant to reduce the activity of the patient's immune system and the risk of organ rejection (marketed by Novartis under the brand names Sandimmune, the original formulation, and Neoral for the newer microemulsion formulation). Multi-phasic micro-components comprising cyclosporine can be used in topical emulsions for treating keratoconjunctivitis sicca, as well.

In certain aspects, the multi-phasic micro-components of the present disclosure comprise one or more of: non-steroidal anti-inflammatory agents (NSAIDs), analgesics, COX-I and II inhibitors, and the like. For example, indomethacin is a suitable NSAID suitable for incorporation into a multiphase micro-component of the disclosure.

As described above, active ingredients can be suitable for use in a wide variety of applications and include proteins, peptides, sugars, lipids, steroids, DNA, RNA, low-molecular weight drugs. The multi-phasic micro-component optionally has such an active ingredient dispersed within one or more phases. For example, such active ingredients can be suspended in a polymer solution or a polymer melt. A first phase can be loaded with an active ingredient or multiple active ingredients. Likewise, a second phase can be loaded with an active ingredient or multiple active ingredients. In some embodiments, the plurality of phases may each contain one or more distinct active ingredients. The phases of the multi-phase composition can also include secondary release systems, such as nanoparticles with sizes equal or smaller than the phase, liposomes, polysomes, or dendrimers. Each of the secondary release systems can be include multiple types of active ingredients, as well, permitting a staging of release of a plurality of active ingredients. The secondary release systems can be formed with the same materials described above in the context of the multiphasic micro-components, however, can be distributed throughout a phase (for example as a continuous and discontinuous phase mixture). Thus, the secondary release system provides an additional amount of control over the release kinetics of active ingredients based and provides an even greater range of complex design and delivery options.

In certain variations, the multiphasic micro-component comprises a conventional active ingredient selected from the group consisting of: a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof. Such active ingredients are further described in U.S. Publication No. 2007/0237800 and in International PCT Application No. PCT/US2010/032971 field on Apr. 29, 2009 entitled “Multiphasic Microfibers for Spatially Guided Cell Growth” to Lahann et al., which are expressly incorporated herein by reference. In certain aspects, the multiphasic micro-component comprises an active ingredient well known to those of skill in the art, such as an active ingredient selected from the group consisting of: low-molecular weight molecules, quantum dots, natural and artificial macromolecules, proteins, sugars, peptides, polypeptides, proteins, amino acids, enzymes, DNA, RNA, polymers, nanoparticles, nano-crystals, growth hormones, growth factors, anti-rejection drugs, anti-inflammatory agents, analgesics, stem cell therapy agents, gene therapy agents, anti-oxidants, free radical scavengers, nutrients, co-enzymes, systemic drugs, therapeutic drugs, localized drugs, tooth whitening agents, skin whitening agents, antimicrobial agents, antibacterial agents, antibiotics, antifungal agents, anti-caries agents, anti-tartar agents, anti-plaque agents, anti-adhesion agents, desensitizing agents, anti-inflammatory agents, malodor control agents, flavoring agents, anti-aging agents, salivary stimulants, periodontal actives, depigmentation agents, skin lightening agents, reflectants, humectants, allergy inhibitors, anti-acne agents, anti-aging agents, anti-wrinkling agents, antiseptics, keratolytic agents, fresheners, healing agents, inflammation inhibitors, wound healing promoters, deodorants, antiperspirants, skin emollients, tanning agents, antifungals, depilating agents, counterirritants, non-steroidal soothing agents, anti-itch agents, poison ivy agents, poison oak agents, burn products, vitamins, cooling agents, heating agents, chelating agents, anti-psoriasis agents, anti-dandruff agents, skin conditioners, moisturizing agents, emollients, humectants, occlusive agents, skin lipid fluidizers, deodorant active agents, antiperspirant active agents, skin and/or scalp sensates, skin and/or scalp soothing and/or healing agents, astringents, opacifying agents, biocides, natural and synthetic extracts and essential oils, nutrients, enzymes, proteins, amino acids, vitamins, analgesics, sunscreen agents, UV absorbers, antioxidants, antibiotics, exfoliants, cell turnover enhancers, coloring agents, sunscreens, nourishing agents, moisture absorbers, sebum absorbers, skin penetration enhancers, colorants, pigments, dyes, flavors, fragrances, and combinations thereof.

In various aspects, the following non-limiting examples describe a shape-shifting process in accordance with the present teachings. A multiphasic micro-component is formed as a polymeric micro-cylinder. In certain preferred embodiments, at least one of the polymers in the micro-component is selected to be a component that is responsive to an external stimulus. Therefore, the micro-cylinders undergo shape-shifting into spheres upon the application of ultrasonication as the external stimulus. While not limiting the present disclosure to any particular theory, it is believed in this particular embodiment that the shape-shifting phenomenon observed is due to heat treatment of the micro-component materials to above their glass transition temperature (T_{g}), along with mechanical perturbation from cavitation and agitation from the ultrasonic energy. Importantly, the multiphasic particles maintain their phase orientation after sonication-induced transformation. By controlling sonication parameters, such as ultrasonic power, frequency, sonication time, and pulsing rates, a wide variety of diverse shape-shifted particles are produced in accordance with the present techniques. Moreover, changing the variables of polymer glass transition temperature, particle aspect ratio, and inner phase orientation, during formation of the micro-components, as well as the dispersion medium for the micro-components, provides the ability to design a variety of unique-shaped particles.

Micro-cylinders having different phases with selective swelling/shrinking characteristics, prepared in accordance with the present teachings, are particularly suitable for biomimetic actuator applications.

Further, the delivery of multiple therapeutic agents with predetermined release rates is particularly advantageous in the area of drug delivery devices. One way of controlling release rate of functional molecules in accordance with the present teachings is to have them disposed in multiphasic particles composed of distinct polymers. By exposing such particles to an external stimulus, such as a change in temperature, pH, or solvent, the micro-components can be controlled to exhibit selective swelling/shrinking or shape-shifting. Such control over micro-components provides the ability to control release rates of multiple drugs/agents from the multiphasic micro-components. Furthermore, these swelling and shrinking features can be used as an actuator since temperature-, pH-, or solvent-activated volume expansion properties can generate biomimetic motion. When these materials exhibit motion in response to biological conditions, they are particularly suitable for use in smart bio-machines or devices.

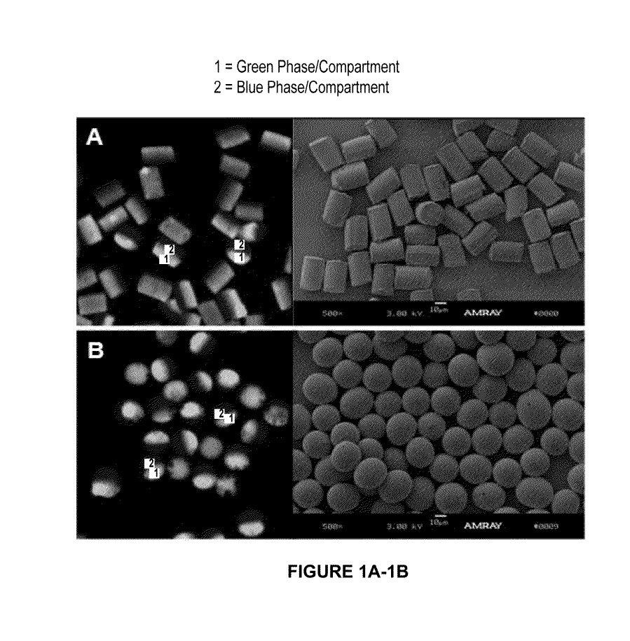

With reference to FIGS. 1A-B, multiphasic micro-components in accordance with certain aspects of the present teachings are in the form of micro-cylinders (shown in FIG. 1A). These micro-cylinders are shape-shifted in accordance with the present teachings to spheres upon exposure to ultrasonication. The biphasic micro-cylinders are formed of poly(lactide-co-glycolide) (PLGA) micro-cylinders. Approximately 10,000 cylindrical particles are suspended in 1 mL of 2 v/v % Tween-20/deionized (DI) water solution. Sonication is performed at room temperature with pulse on for 9 seconds and off for 5.5 seconds. The total time for complete shape-shifting is about 2 minutes.

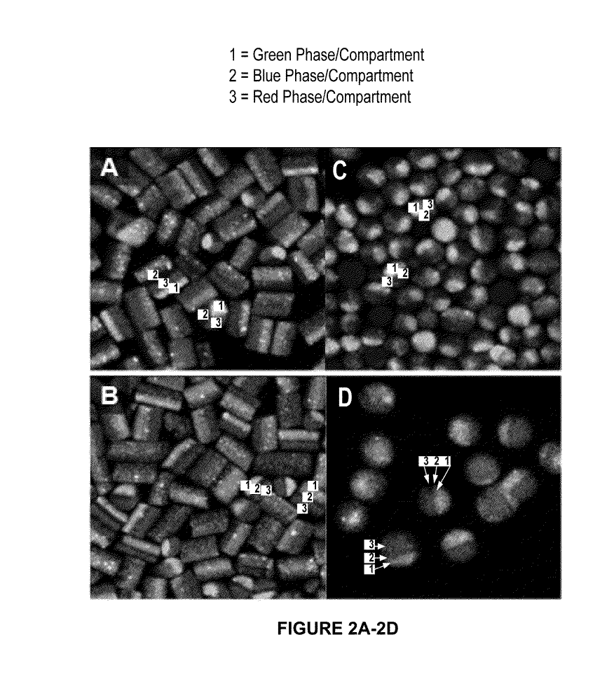

FIGS. 2A-2B show CLSM micrographs of triphasic PLGA micro-components. In FIG. 2A, the micro-components are cylinders with a pie-shaped orientation of the respective three phases. The cylinders are shape-shifted into spheres (where the three phases are of a stripe-type) upon ultrasonication, as shown in FIG. 2B. Notably, the shape-shifted spheres retain their phase orientation and alignment upon ultrasonication for 2 min in 2 w/v % Tween-20/deionized (DI) water.

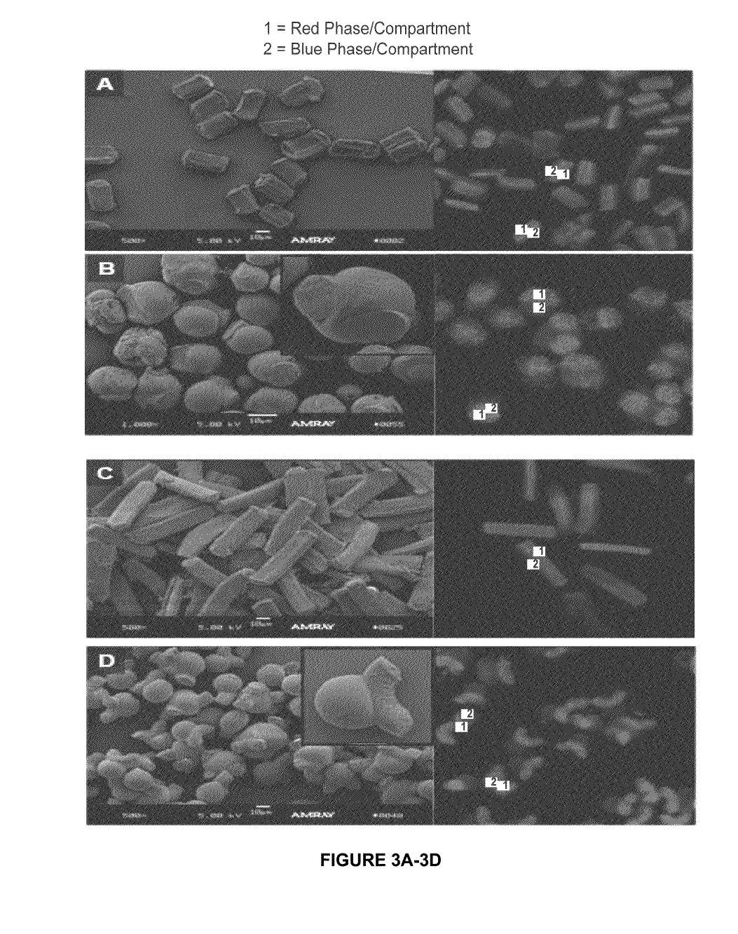

FIGS. 3A-D show CLSM and SEM micrographs of biphasic PLGA/poly(methyl methacrylate) (PMMA) cylinders. The cylinders in FIG. 3A have a 20 μm length, while those in FIG. 3C have a 50 μm length. After exposing the micro-components cylinders to an external stimulus in the form of ultrasonication, the 20 μm cylinders in FIG. 3A shape-shifted as shown in FIG. 3B. Likewise, FIG. 3D shows the shape-shifting of the 50 μm cylinders in FIG. 1C upon exposure to ultrasonication. Due to the difference of glass transition temperature between PLGA (45-50° C.) in the first phase and PMMA (105° C.) in the second phase, the first PLGA phase transforms its shape to sphere, while the second PMMA phase maintains the original shape.

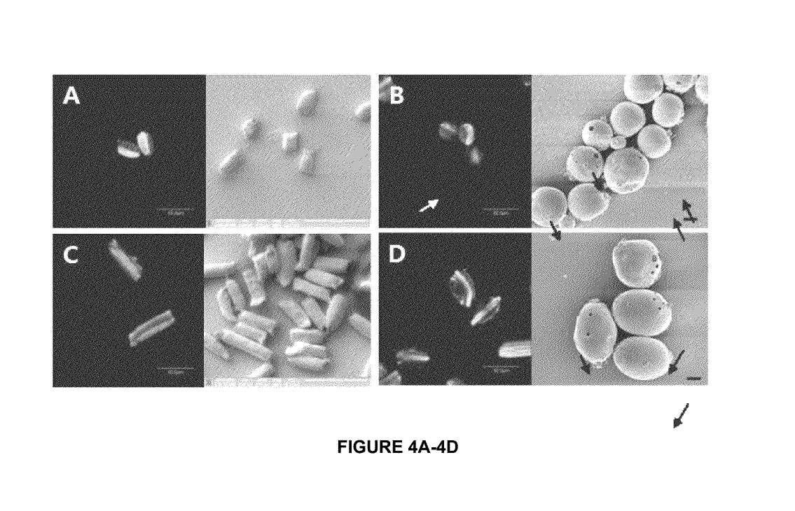

FIGS. 4A-D show CLSM and SEM images of multiphasic micro-components in the shape of micro-cylinders, where a first phase comprises pure PLGA and the other second phase comprises a mixture of PLGA and poly(vinyl cinnamate) (PVCi) having a 1:1 ratio. The PVCi in the second phase is photocrosslinked before applying ultrasonication. The cylinders have a 20 μm length in FIG. 4A and a 50 μm length in FIG. 4C. FIG. 4B shows the shape-shifting of the 20 μm cylinders in FIG. 4A upon ultrasonication. FIG. 4D likewise shows the shape-shifting of the 50 μm cylinders in FIG. 4C upon ultrasonication. Notably, the crosslinked PVCi in the second phase maintains its morphology during the shape-shifting process. After shape-shifting, fragments are responsible for PVCi observed on the PLGA sphere (indicated as arrows in B and D).

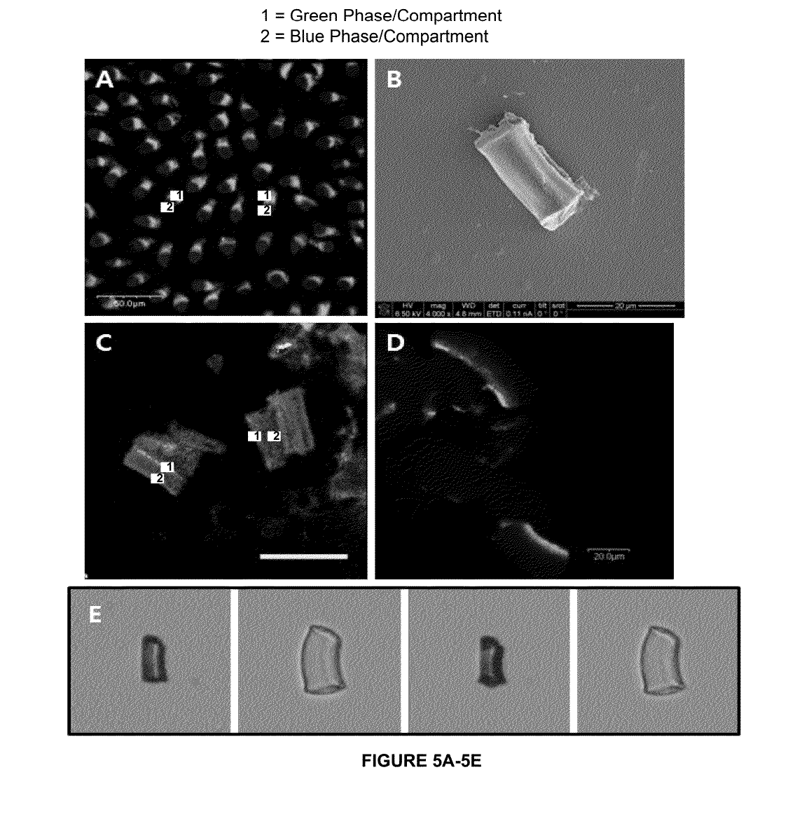

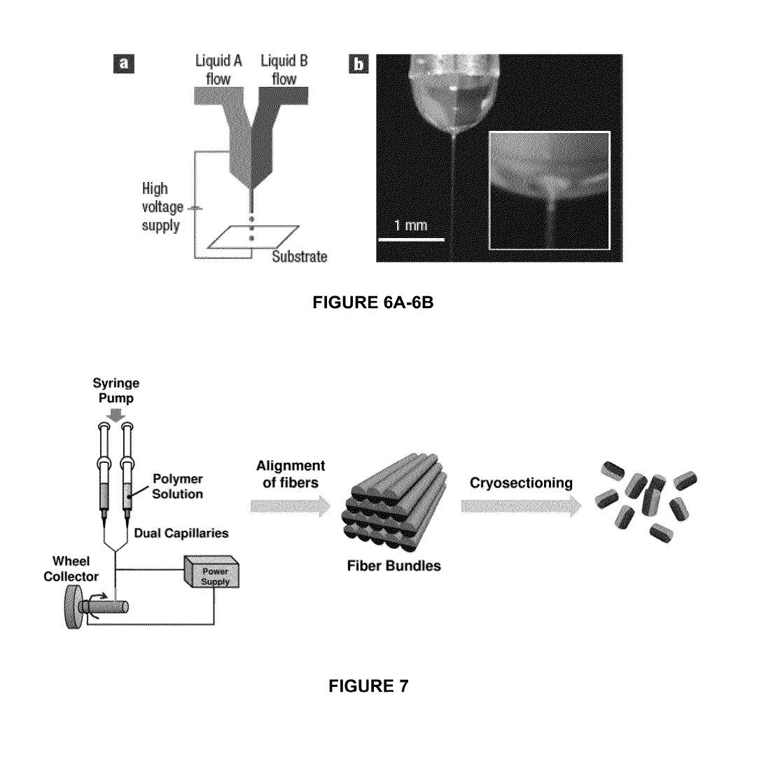

FIGS. 5A-E show another embodiment of shape-shifting in accordance with the present disclosure. FIG. 5A shows a cross-sectional CLSM image of a biphasic micro-cylinder, comprising a first phase composed of PVCi (blue phase) and a second phase comprising PEO (green phase). FIG. 5B is an SEM image of a sectioned biphasic fiber with 20 μm in length. FIG. 5C is a CLSM image of a plane view of a biphasic micro-cylinder after photocrosslinking. FIG. 5D shows a shape-shifted micro-cylinder created by introducing dioxane as a stimulator chemical/molecule. Because photocrosslinked PVCi is largely swelled by dioxane in the first phase, as compared to PEO in the second phase, the micro-cylinder is bent toward second (PEO) phase. As shown in CLSM, the first PVCi phase (Blue part) is enlarged when the dioxane is introduced as an external stimulus. FIG. 5E depicts an optical microscopy image of a 20 μm micro-cylinder showing reversible switching. When the dioxane is removed (via drying), the micro-cylinder returns to its original shape and therefore the physical deformation/shape-shifting is reversible.

### Example 1

### Preparation of Multiphasic Microcylinders

A variety of multiphasic microparticles can be prepared by the electrohydrodynamic jetting procedures described previously above. Such microparticles can include a variety of distinct shapes, including microcylinders, microdisks, and microfibers. Precise engineering of compartmentalized microparticles, such as cylinders or disks, with a wide range of properties is achieved by adjusting a number of experimental parameters during electrohydrodynamic (EHD) co-jetting, including polymeric concentrations and applied voltages.

In this example, electrohydrodynamic co-Jetting is employed in this example to form a variety of distinct multiphasic microcylinders. Two or more different jetting mixtures are prepared for each anisotropic microfiber production. Herein, different anisotropic microfibers are produced from various combinations of polymeric materials.

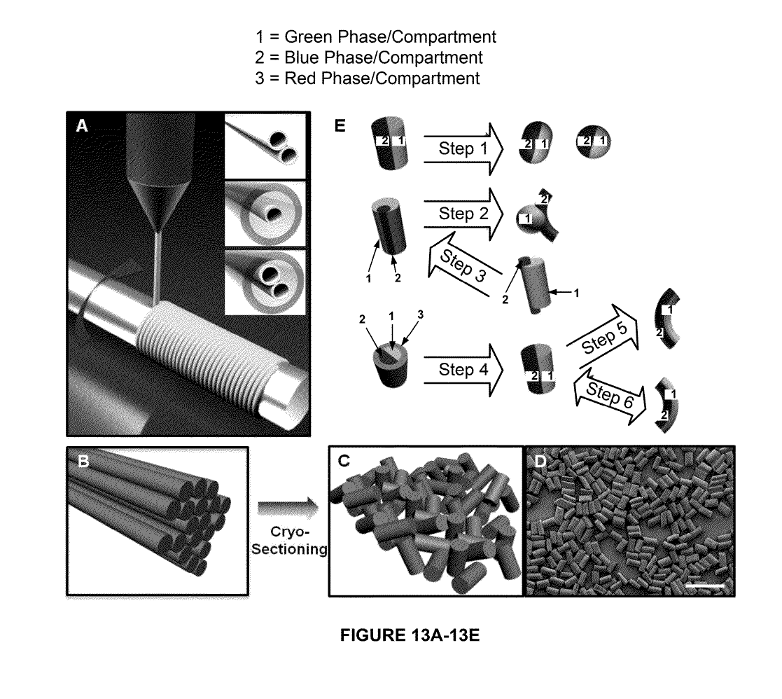

The general concept and experimental set-up is shown in FIGS. 7 and 13. A plurality of multiphasic anisotropic particles receptive to an external stimulus are prepared by use of an electrohydrodynamic (EHD) co-jetting system for continuously forming multiphasic fibers, where well-aligned fibers are collected on a rotating wheel collector. FIGS. 7 and 13A. FIG. 13B shows a bundle of the multiphasic fibers formed in FIG. 13A. In FIG. 13C, micro-sectioning of the fiber bundles from FIG. 13 B with a cryogenic sectioning process forms a plurality of microcylinder particles having preselected dimensions (e.g., length). After sectioning of the fibers, micro-cylinders are formed with substantially the same diameters and having well-defined and controllable lengths, as shown in the SEM image of typical microcylinders in FIG. 13D. Moreover, microcylinders with multiple, different compartments are prepared using a range of different needle sets in the electrohydrodynamic jetting process of FIG. 13A (see insets in FIG. 13A, including core/shell and dual-core/shell needle arrangements). FIG. 13E is an overview of shape reconfiguration techniques based on different embodiments of multicompartmental multiphasic anisotropic microcylinders according to the present technology, specifically: (i) shape-shifting, (ii) reversible switching, and (iii) three-way toggling.

All polymers used herein include poly(DL-lactide-co-glycolide) (PLGA) (85:15, M_{w}=50-75,000 g/mol), poly(methyl methacrylate) (PMMA) (M_{w}=350,000 g/mol), poly(vinyl cinnamate) (PVCi) (M_{w}=200,000 g/mol), polystyrene (PS) (M_{w}=280,000 g/mol), poly(ethylene oxide) (PEO) (M_{w}=100,000 g/mol), Poly(ethylene glycol) (PEG) diglycidyl ether (Mₙ=526 g/mol), Polyethylenimine (PEI) (branched, M_{w}=25,000 g/mol), are purchased from Sigma-Aldrich, USA. The fluorescence dyes poly[(m-phenylenevinylene)-alt-(2,5-dibutoxy-p-phenylenevinylene)] (MEHPV) and poly[tris(2,5-bis(hexyloxy)-1,4-henylenevinylene)-alt-(1,3-phenylenevinylene)] (PTDPV), which are used as CLSM markers with blue and green emission, are purchased from Sigma-Aldrich, USA. The red-emitting dye ADS306PT is purchased from American Dye Source, Canada. The solvents chloroform, 1,4-dioxane and N,N-dimethylformamide (DMF) are purchased from Sigma-Aldrich, USA and used without further purification.

The preparation of PLGA polymeric solutions for EHD co-jetting follows literature-described procedures. Typically, 30 w/v % of PLGA and a trace amount of appropriately selected fluorescence dyes are dissolved in a solvent mixture of chloroform and DMF (95:5, v/v). The experimental setup contains a syringe pump (Fisher Scientific, Inc., USA), a power supply (DC voltage source, Gamma High Voltage Research, USA), and a rotary collector (Synthecon, Inc., modified to experimental requirements). The polymer solutions are delivered at a constant flow rate of 0.05 ml/h via vertically positioned 1 mL syringes equipped with 26 G needles (Hamilton Company, USA). A driving voltage of 11.1 kV is applied to the metal needles. Stable fibers are collected at a tip-to-ground distance of approximately 5 cm.

The fiber bundles are vacuum-dried overnight. Next, the fibers are micro-sectioned to form microcylinders. Microsectioning of the fiber bundles is done using a cryostat microtome (Microm HM550, Thermo Fisher Scientific, Inc., Germany). The samples are embedded into a sectioning medium (Tissue-Tek O.C.T. Compound, Andwin Scientific, USA), cooled to −20° C., and microsectioned at a desired length. After washing with deionized (DI) water, the microcylinders are separated using microfilters (Spectra Mesh Woven Filters, Spectrum Laboratories, Inc., USA) to obtain monodisperse particles.

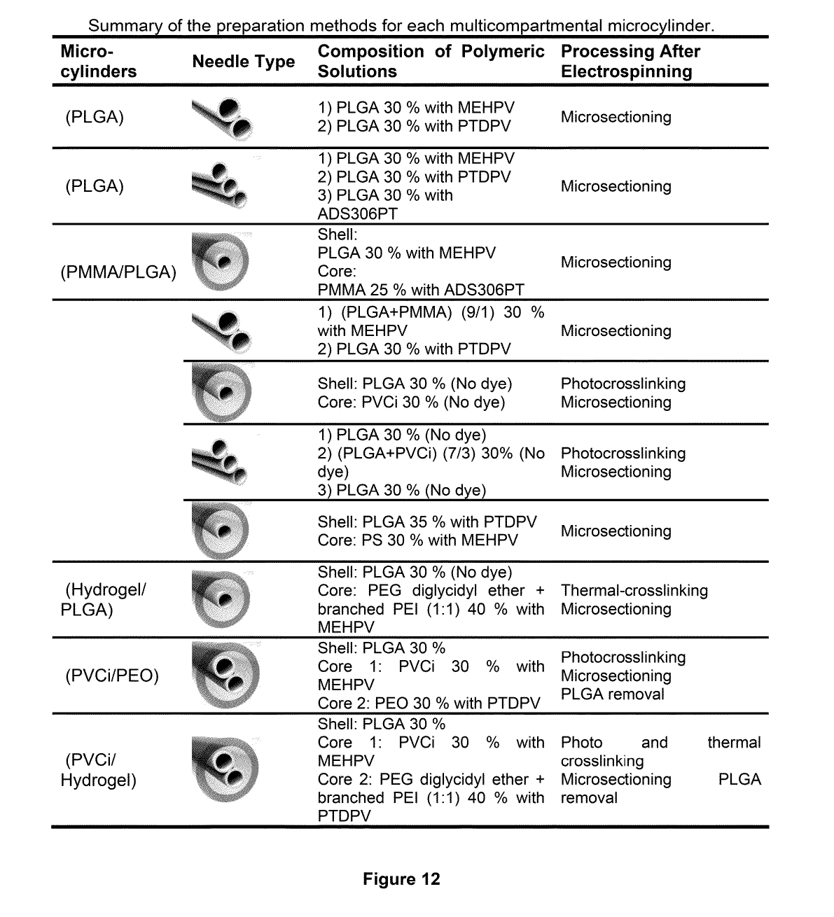

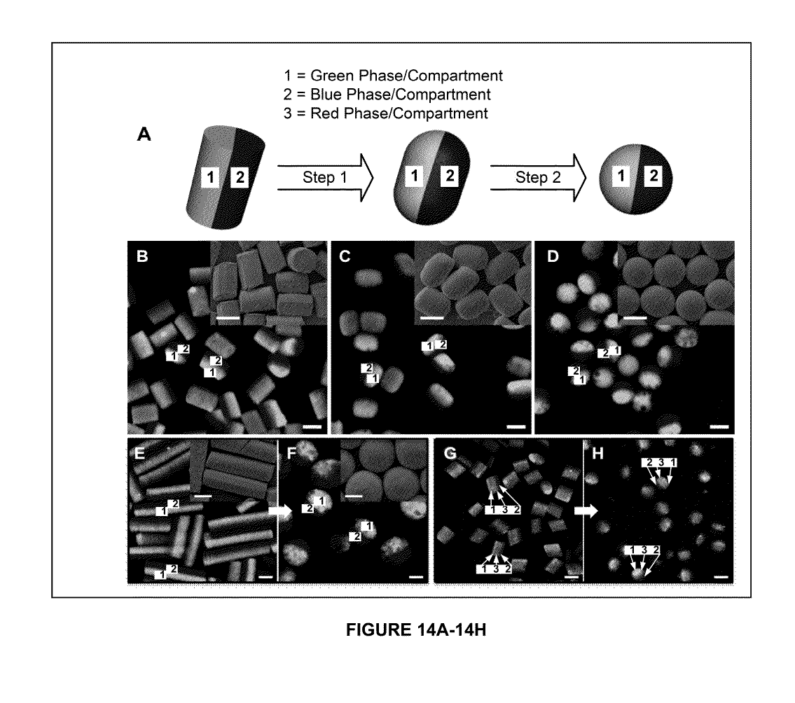

To obtain the different types of multiphasic microcylinders the procedures described just above are modified in so far as the needle arrangement during electrohydrodynamic co-jetting is altered. To obtain aligned fiber bundles with compositionally dissimilar compartments, a PLGA shell stream is employed that encapsulates a central “core” of the fiber. FIG. 12 summarizes the needle arrangements, material compositions, and preparation procedures to achieve the different multiphasic microcylinder particles (generally shown in FIGS. 14-15).

Fluorescence images of particles (CLSM images) are visualized using an Olympus FluoView 500. Three different lasers, 405 nm laser, 488 nm Argon laser, and 533 nm Helium-Neon green (HeNeG) laser, are used to excite the dyes (MEHPV, PTDPV, and ADS306PT), respectively. The barrier filters are set to 430-460 nm for MEHPV, 505-525 nm for PTDPV, and 560-600 nm for ADS306PT. The glass transition temperatures of PLGA and PMMA are evaluated based on their DSC thermograms. The measurements are carried out using a Perkin-Elmer DSC-7 at a scanning rate of 5° C./min.

Each polymer is kept in aluminum pans and an empty pan is used as the reference. In order to obtain SEM images, the samples are coated with gold before analysis and the particle morphology is examined using an AMRAY 1910 Field Emission Scanning Electron Microscope (FEG-SEM) and FEI Nova Nanolabs. Fourier transformed Infrared (FTIR) spectra of KBr pellets are obtained using a Nicolet 6700 spectrometer. Fluorescence optical microscopy (OM) images are collected with a Nikon, Eclipse 80i.

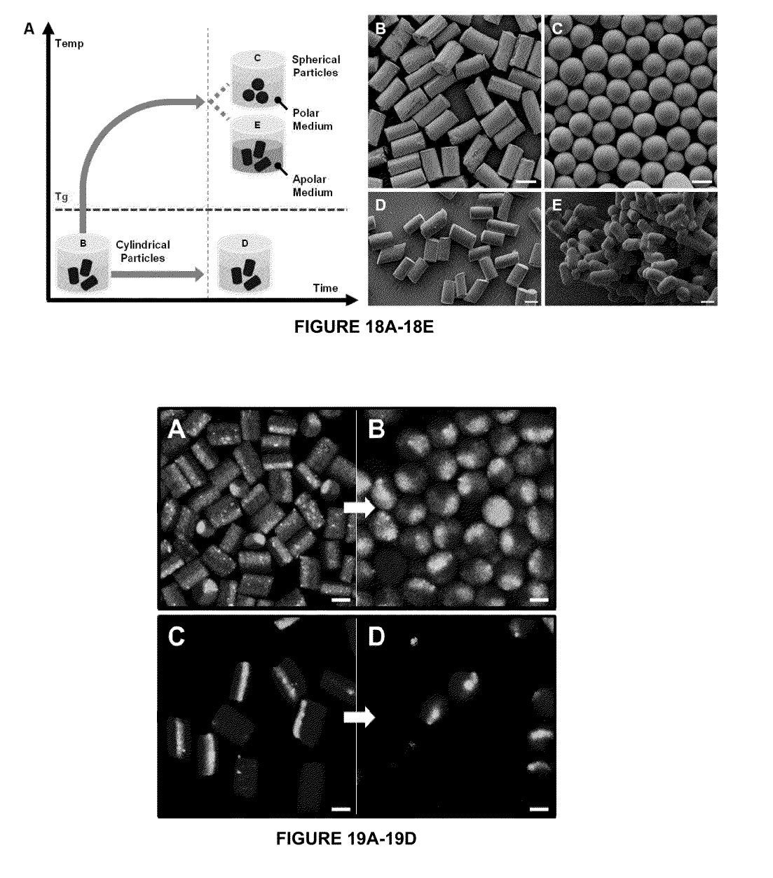

Shape-shifting of multiphasic microcylinders is performed by applying an external stimulus to which the microcylinder is physically responsive (and transforms by shape or volume), such as ultrasound-mediated heat treatment. When relatively hydrophobic PLGA microcylinders are treated with ultrasound in water, the particle temperature increased above the glass transition temperature T_{g} of polymer (T_{g}=47-48° C.), and the cylindrical particles are reconfigured into spheres due to the minimization of surface-to-volume ratio (FIG. 18). Only after a short period, all the cylinders are converted to spheres, and the inner compartments are fully retained in the particles after shape-shifting. Moreover, polymers with different properties in response to temperature are introduced to produce unique shaped particles. Typical shape-shifting process with ultrasound treatment is as follows. Approximately 10,000 cylinders are dispersed in 1 mL of medium in an eppendorf tube and treated with ultrasound (Ultrasonic Processor, Cole-Parmer, USA) at room temperature. Medium used for the ultrasound treatment is either 2 v/v % Tween 20/DI water or a mixture of ethanol and 2 v/v % Tween 20/DI water (1:1, v/v) and duration time is varied depending on the desired shape with pulse on for 9 sec and off for 5.5 sec in all cases. The detailed experimental conditions (including sonication medium and duration) used to treat each multiphasic microcylinder are listed below in Table 1.

TABLE 1
Summary of the shape-shifting process for multiphasic microcylinders.

| | | | Sonication |
|---|---|---|---|
| | Cylinder | | Duration |
| Micro- | Length | | Time |
| Cylinders | (μm) | Sonication Medium | (min) |
| FIG. 14C | 30 | 2 v/v % Tween 20/DI water | 1.5 |
| FIG. 14D | 30 | 2 v/v % Tween 20/DI water | 3 |
| FIG. 14F | 70 | 2 v/v % Tween 20/DI water | 5 |
| FIG. 14H | 70 | 2 v/v % Tween 20/DI water | 3 |
| FIG. 15C | 50 | Ethanol:(2 v/v % Tween 20/ | 1 |
| | | DI water), 1:1 (v/v) | |
| FIG. 15D | 50 | Ethanol:(2 v/v % Tween 20/ | 1.5 |
| | | DI water), 1:1 (v/v) | |
| FIG. 15E-15H | 200 | 2 v/v % Tween 20/DI water | 2 |
| (from left to right) | 50 | 2 v/v % Tween 20/DI water | 5 |
| | 70 | Ethanol:(2 v/v % Tween 20/ | 3 |
| | | DI water), 1:1 (v/v) | |
| | 10 | Ethanol:(2 v/v % Tween 20/ | 1 |
| | | DI water), 1:1 (v/v) | |

FIG. 18 also shows switching (or reconfiguration) of polymeric microcylinders into microspheres. A graphical representation in FIG. 18A shows temperature versus time for an exemplary shape-shifting or reconfiguration process. When polymeric microcylinders are treated with ultrasound, the increase in temperature above the T_{g} of the polymer causes cylindrical particles (18B) to take on a spherical envelope (18C). As control experiments, the microcylinders are treated with ultrasound in an ice bath (18D), and in an heptane (bₚ=98° C.) (18E). An ice bath is used for keeping the temperature below T_{g} of polymer during sonication, and heptane is used for the apolar medium, which does not have the driving force to minimize the surface area of the particles. In both cases, no change in particle shapes is observed.

Shape-Shifting of Compartmentalized Microcylinders

An important aspect of particle reconfiguration/switching principle of the present teachings is that it is applicable to a variety of diverse microparticles. Thus, in FIG. 19, a wide-variety of cylinders are shape-shifted. First, a broad range of compartmentalization patterns including two types of tricompartmental (side-by-side (FIG. 14G) and pie-shape (FIG. 19A)), and heptacompartmental cylinders (FIG. 19C) are investigated. Upon ultrasound treatment, the pie-shaped tricompartmental (19A) and heptacompartmental (19C) microcylinders are reconfigured to microspheres, 19B and 19D, respectively. CLSM and OM images show the heptacompartmental particles with one compartment having green (PTDPV) and the others having blue (MEHPV) are successfully confined in one compartment.

The PLGA microcylinders in FIG. 19A are prepared with pie-shaped three needle configuration. The compositions of polymeric solutions are identical with that of FIG. 14G, including polymer concentrations and fluorescence dyes for visualization, except for the needle geometry. In case of FIG. 19C, seven-needle configuration is adapted; six needles (blue) are surrounding one needle (green). The polymer concentrations introduced in each needle is same with that of FIG. 14B. See also, FIG. 12.

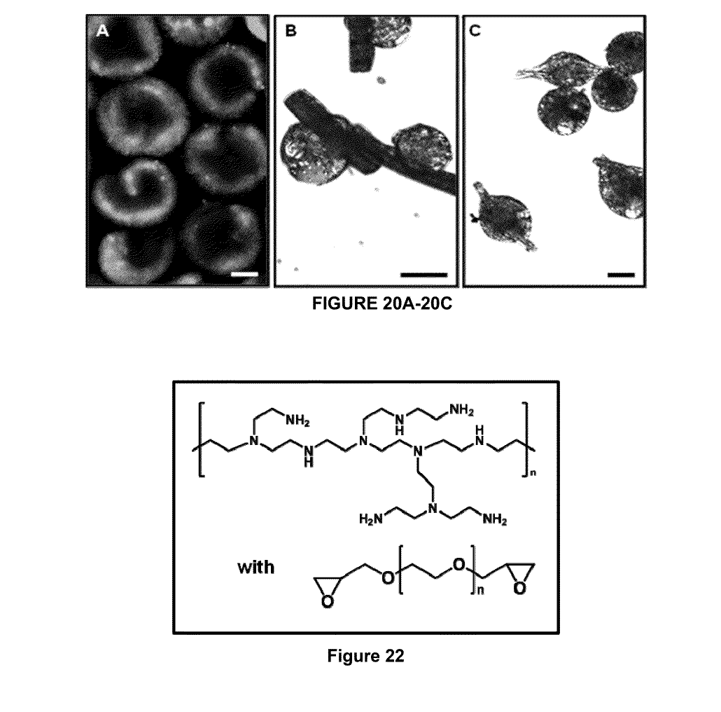

More examples of particle reconfiguration are displayed in FIG. 20, where anisotropic shape-shifting of bicompartmental microcylinders after ultrasound treatment. In FIG. 20, the shape shifting is of an anisotropic nature. Various microcylinders including bicompartmental PLGA/(PLGA+PMMA), PVCi/PLGA, and tricompartmental PLGA/(PLGA+PVCi)/PLGA are shape-shifted upon ultrasound treatment, and each inner structure of the multiphasic particles is demonstrated by CLSM images. Particles in FIGS. 20A, 20B and 20C correspond to the SEM images of FIGS. 15E, 15F, and 15G respectively.

Free Surface Energy Simulations

FIG. 21 is a comparison of experimentally obtained shape-shifted particles with expected equilibrium envelopes by Surface Evolver computation. Comparison of experimentally obtained shape-shifted particles with expected equilibrium envelopes. CLSM and the corresponding models show bicompartmental PLGA/PLGA (A-C) and PVCi/PLGA (D-F) microcylinders after evolving into spheres. Evolving from a defined initial structure, the Surface Evolver models the surface toward minimal energy profile. The unique changes of particle morphologies are thus theoretically predicted with a Surface Evolver (FIG. 21) and correspond to empirical observation. CLSM and the corresponding models show bicompartmental PLGA/PLGA (21A-21C) and PVCi/PLGA (21D-21F) microcylinders after evolving into spheres. Notably, FIGS. 21A-21C show an isotropic transformation for a biphasic/bicompartmental particle, while FIGS. 21D-21F show an anisotropic transformation for an alternative embodiment of a biphasic/bicompartmental particle.

The simulated equilibrium states for bicompartmental PLGA/PLGA (FIG. 21A-21C) microcylinders, thereby evolved into spheres and show good agreement with that of experimentally observed particle shapes. Given one compartment with no surface tension energy in the software, the simulation provided the selective changes in particle shape, and closely resembled bicompartmental PVCi/PLGA microcylinders (FIG. 21). A crosslinked PVCi maintains its morphology as a cylindrical shape since the compartment had no driving force to shape-shift due to its rigid property and high stability to temperature.

### Example 2

Bicompartmental Hydrogel/PLGA microcylinders comprising a hydrogel in a first phase and PLGA in a second phase are prepared with core/shell needles like the electrohydrodynamic jetting methods described in the context of Example 1. A thickness of each respective phase/compartment can be readily controlled by adjusting the flow rates through the needles for the core and shell, respectively. In this example, the hydrogel/PLGA microcylinders are prepared with core and shell flow rates of 0.01 and 0.05 mL/hr, respectively.

The chemical structure of the hydrogel is illustrated in FIG. 22. PEG diglycidyl ether and branched PEI is mixed with 1:1 (w/w) ratio. The amine groups in PEI and epoxide groups in PEG are crosslinked to form hydrogel. After the jetting, the microfiber is loaded into vacuum desiccators for 24 hrs at room temperature for a complete removal of the organic solvent. During this step, crosslinking of hydrogel is performed simultaneously. Approximate swelling ratio of the hydrogel is 280% in deionized (DI) water (obtained from bulk films of the hydrogel).

### Example 3

Bicompartmental hydrogel/PLGA microcylinders comprising an organogel comprising PVCi and PEO in a first phase and a PLGA as a distinct phase. The microcylinders are prepared with core/shell needles like the electrohydrodynamic jetting methods described in the context of Example 2, except that a configuration with dual-core and shell needles is used. PLGA solution is introduced into the shell needle with a flow rate of 0.05 mL/hr. PVCi and PEO are delivered into the each core needle with variable flow rates (0.02 mL/hr for FIG. 4E).

In embodiments where a shell stream is used during electrohydrodynamic co-jetting in a configuration with two or more core materials, such as the PLGA shell stream, simultaneous processing of substantially dissimilar materials is possible (to form them together as a dual-core structure). Subsequent removal of the sacrificial shell results in microcomponents with substantially different compositions in the different compartments, thus permitting a broad diversification of the compartment compositions.

After jetting, the PVCi compartment is photocrosslinked. Then, the PVCi/PEO cylinders are prepared by microsectioning, followed by the removal of PLGA sacrificial shell phase/compartment.

The chemical structure of PVCi (before and after photocrosslinking) and PEO used as core materials in dual-core/shell jetting is provided in FIG. 23.

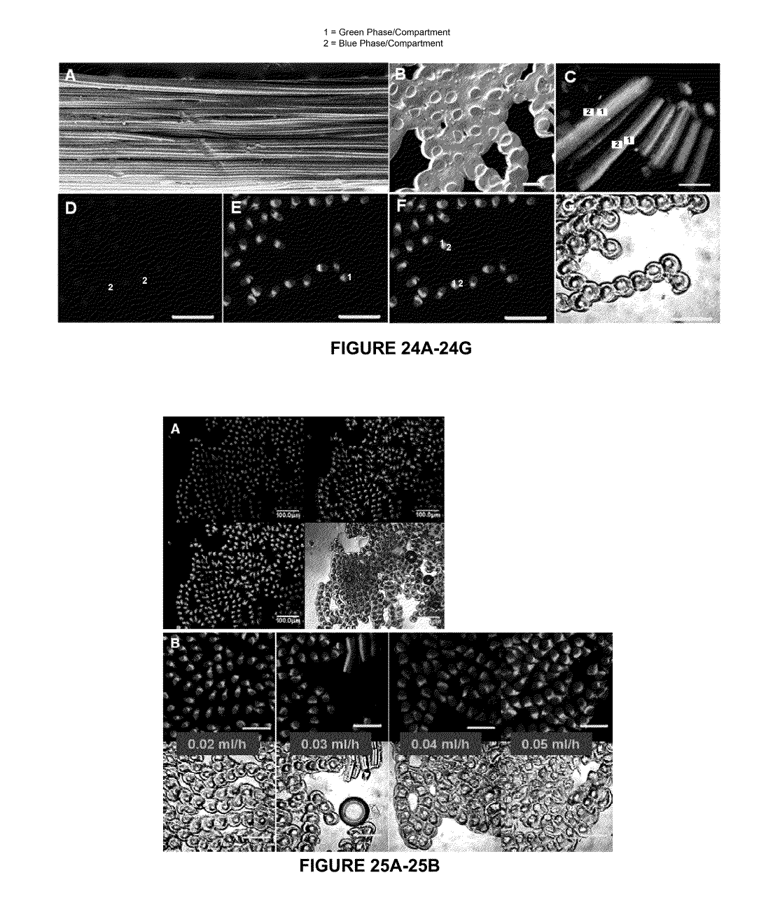

FIG. 24 shows SEM and CLSM images of the PVCi/PEO@PLGA dual-core/shell fibers (formed from flow rates of core and shell: 0.02 mL/hr and 0.05 mL/hr, respectively). From the cross-sectional view, each fiber contains PVCi and PEO as a dual-core. The 3D CLSM images also confirm that each fiber has bicompartmental cores. Blue and green emission comes from the fluorescence dyes MEHPV and PTDPV, which are incorporated into the PVCi and PEO, respectively. Green and blue colorants (representing PEO and PVCi respectively) are indicated as 1 or 2. There is no fluorescent dye in PLGA shell compartment. Sharp boundaries between blue and green colors as well as core and shell compartments demonstrate the consistency of compartmentalization during the preparation of microcylinders.

FIGS. 24D-24G show cross-sectional views of microfibers of certain embodiments of the present technology comprising distinct phases or compartments of PVCi, PEO, and PLGA having dual core phases surrounded by a shell phase, overlaid with CLSM and differential interference contrast (DIC) images. A bicompartmental dual-core is clearly observed in a PLGA shell phase, as reflected by FIG. 24G.

FIGS. 25A-25B show CLSM and DIC cross-sectional images of certain embodiments of the present disclosure, where microfibers comprise distinct phases or compartments with an arrangement of dual core phases (comprising PVCi and PEO, respectively) and a shell phase surrounding the dual core (comprising PLGA). FIG. 25A is a low magnification of a cross-sectional view (core flow rate: 0.02 ml/h), which indicates the suitability to scaling-up such a process for mass production. FIG. 25B shows cross-sectional views of the microfibers formed as a function of core flow rates varying from 0.02 to 0.05 ml/h for a shell flow rate of 0.1 ml/h. The thickness of core is controlled by adjusting a core flow rate between 0.02 to 0.05 mL/hr.

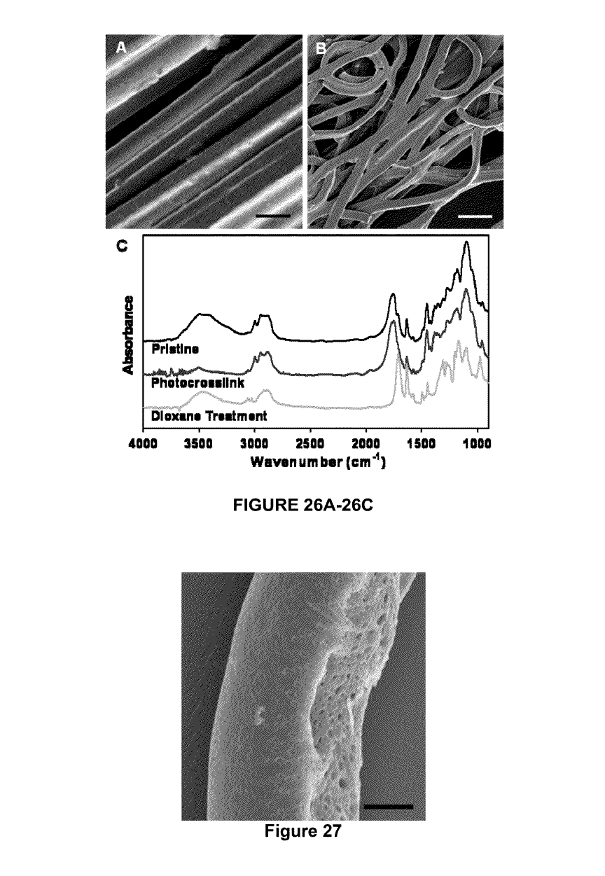

After photocrosslinking and the removal of PLGA, bicompartmental PVCi/PEO microfibers can be isolated as shown in FIG. 26. FIG. 26A is an SEM image of as-prepared PVCi/PEO@PLGA microfibers. FIG. 26B shows bicompartmental PVCi/PEO microfibers after photocrosslinking of PVCi and removing the PLGA shell. FTIR analysis of the fibers after jetting, photocrosslinking, and dioxane treatment is provided in FIG. 26C. Before removing the PLGA (black and red spectra), FTIR spectra show the main characteristic bands for PLGA and PVCi, including C═O bands of PLGA at 1754 cm⁻¹, C—H bands of polymer backbone at 2800 about 3000 cm⁻¹, C═C bands of the cinnamate groups at 1631 cm⁻¹ and C═O stretches of unsaturated ester groups in PVCi at 1716 cm⁻¹. After crosslinking, the band for C═C is reduced and the band for unsaturated ester groups move to higher wavenumber, resulting in overlapping with C═O band of PLGA. After removing the PLGA, characteristic bands for the crosslinked PVCi and PEO can be detected (green spectrum in FIG. 26C).

When PVCi/PEO bicompartmental microcylinders are treated with DI water, water soluble PEO is dissolved and thus hemisphere-microcylinders can be obtained, as shown in the SEM image in FIG. 27.

Reversible Shape-Switching

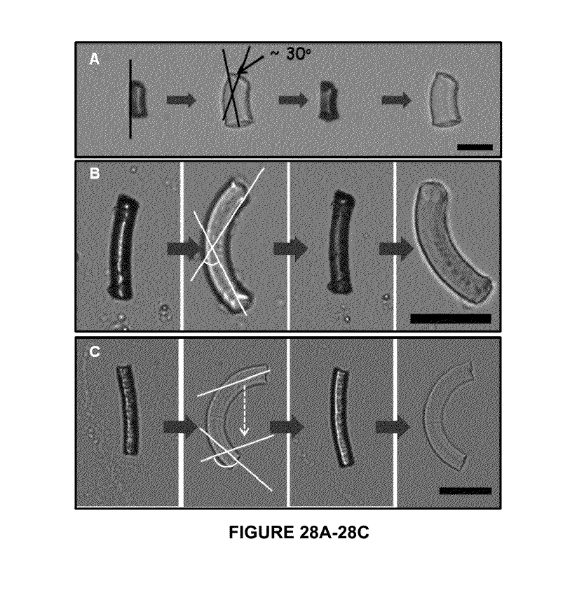

FIG. 28 shows the reversible actuation of bicompartmental PVCi/PEO microcylinders with different lengths. FIG. 28 is an OM of bicompartmental PVCi/PEO microcylinders, 20 μm (28A) 50 μm (28B) and 100 μm (28C) in length and shows various actuation angles. The PVCi/PEO microcylinders are loaded on a glass substrate, and solvent-responsive actuation is observed using an optical microscope as following. FIG. 28 thus shows reversible actuation. The actuation behavior is observed by sequential introduction of dioxane, and the actuation angle of each cylinder is measured.

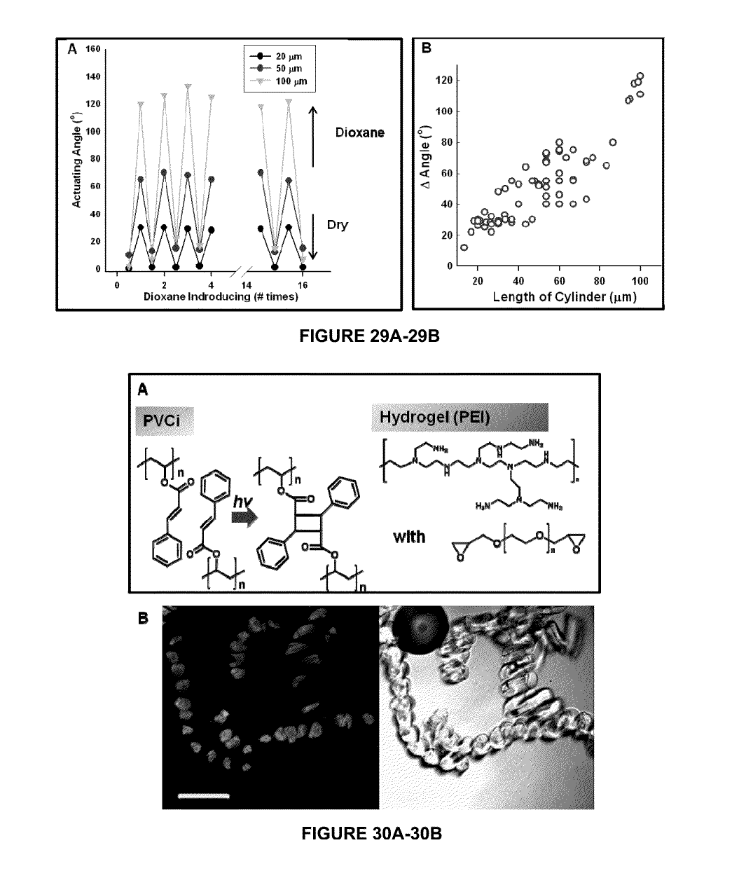

The reversibility can be maintained over 10 times in repeating experiments (FIG. 29A), and the actuation angle difference (A angle: between a dry state and a dioxane state) have a linear relationship with microcylinder lengths (FIG. 29B).

### Example 4

Bicompartmental hydrogel/PVCi microcylinders comprising a hydrogel in a first phase, and PVCi in a second phase and a PLGA as a distinct third shell phase. The microcylinders are prepared with core/shell needles like the electrohydrodynamic jetting methods described in the context of Example 3 with dual-core and shell needles is used.

The hydrogel adapted is based on PEI (50% w/w), which is pH-responsive hydrogel. PEI experiences a greater amount of swelling in lower pH environments. The PVCi and hydrogel structures are shown in FIG. 30A. Fluorescence dyes blue (MEHPV) and green (PTDPV) are incorporated in PVCi and hydrogel, respectively. PLGA solution is introduced into the shell needle with a flow rate of 0.05 mL/hr. PVCi and PEO are delivered into each core needle with variable flow rates (0.02 mL/hr for FIG. 16E). After photo- and thermal crosslinking of PVCi/Hydrogel@PLGA dual-core/shell microfibers, PVCi/Hydrogel microcylinders are prepared by microsectioning and the removal of PLGA shell compartment. FIG. 30B are cross-sectional CLSM and DIC images of PVCi/Hydrogel@PLGA microfibers thus formed.

FIG. 33 represents bicompartmental PVCi/Hydrogel microcylinders showing shape-switching behaviors in response to pH, resulting in different actuation. The actuation angle of the bicompartmental microcylinders increases by 20% when the environment is changed from DI water to pH 4 buffer solution (approximately, 100° to 120°) due to the increment of the swelling of hydrogel compartment. In addition, the hydrogel compartment is thicker in pH 4 than in DI water.

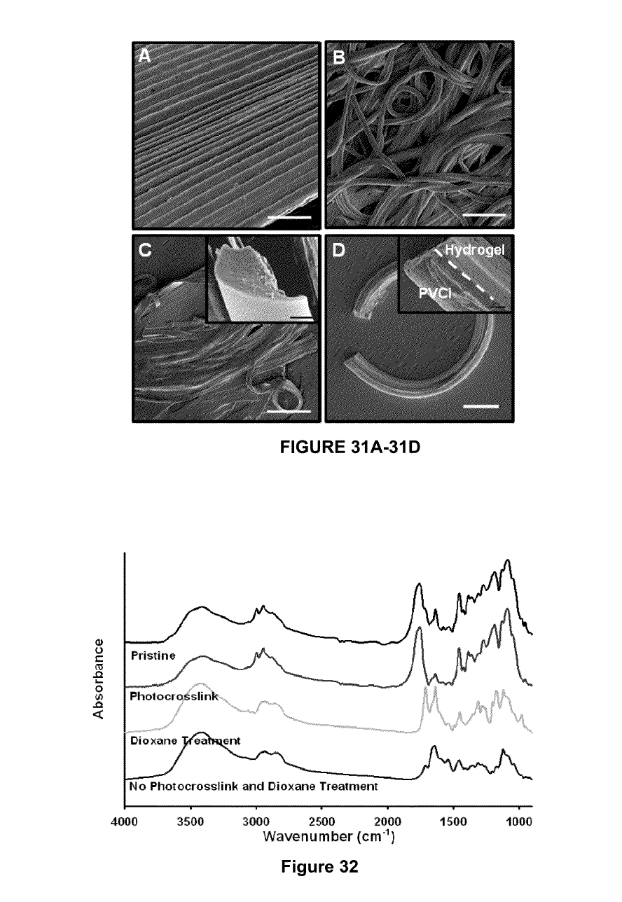

FIG. 31 is an SEM image of the as-prepared PVCi/Hydrogel@PLGA microfibers. FIG. 31B is an SEM image of PVCi/Hydrogel microfibers after photocrosslinking of PVCi, thermal crosslinking of the hydrogel and the PLGA dissolution. FIG. 31C shows hydrogel microfibers after thermal crosslinking of the hydrogel compartment only (without photocrosslinking) and the PLGA dissolution. An inset shows a cross-sectional view of hemisphere-cylinder morphology. FIG. 31D shows PVCi/Hydrogel microcylinders, 200 μm in length.

FIG. 31 shows SEM images of the as-prepared microfibers (before dissolution of PLGA). After photo- and thermal crosslinking of the fibers, bicompartmental PVCi/Hydrogel microcylinders can be isolated by removing the PLGA shell compartment as shown in FIG. 31B. To demonstrate successful crosslinking of the hydrogel compartment, only PEO is thermally crosslinked and hemisphere-microcylinder formation is observed (FIG. 31C). FIG. 31D presents SEM images of bicompartmental PVCi/Hydrogel microcylinders with a length of 200 μm. Two different surface morphologies are observed between convex and concave compartments in the microcylinders as shown in higher magnification SEM images (inset of FIG. 31D). Because the images are taken after drying the microcylinders from dioxane, the outer surface (convex compartment) is the organogel (PVCi).

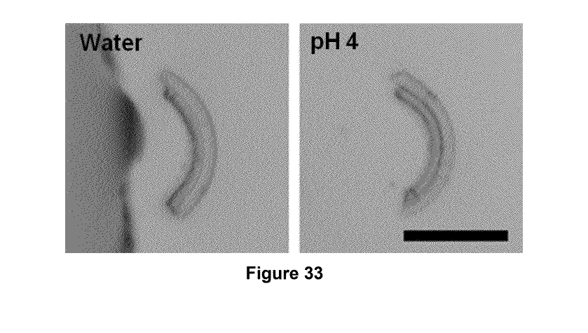

FIG. 32 shows FTIR spectra of as-prepared PVCi/hydrogel@PLGA microfibers (black), after photocrosslinking (red) and the PLGA removal (green). As a control experiment, FTIR spectrum of the hydrogel microfibers which is obtained by PLGA removal without photocrosslinking is also provided (the blue spectrum is the hydrogel). Similar to PVCi/PEO@PLGA microfibers, the most dominant characteristic bands are PLGA before solvent treatment. After photocrosslinking, both C═C band and shifting C═O for unsaturated ester bonds are decreased. When the polymer fibers are exposed to dioxane after photocrosslinking, the characteristic FTIR spectrum of PVCi and PEI can be obtained. As presented in green spectrum, all characteristic bands for PLGA are reduced, resulting in almost identical FTIR spectrum with green spectrum of FIG. 26C, except for 1650 cm⁻¹ for primary amine N—H band, attributable to PEI. On the other hand, without photocrosslinking, the FTIR spectrum of PEI are obtained without PVCi characteristic bands after solvent treatment, as shown in the blue spectrum.

FIG. 33 shows pH-Response of bicompartmental PVCi/Hydrogel microcylinders (OM images of bicompartmental PVCi/Hydrogel microcylinders) in different pH conditions. As noted above, the hydrogel is based on PEI (50% w/w), which is a pH-responsive hydrogel. FIG. 33 represents bicompartmental PVCi/Hydrogel microcylinders showing shape-switching behaviors in response to pH, resulting in different actuation. The actuation angle of the bicompartmental microcylinders increases by 20% when the environment is changed from DI water to pH 4 buffer solution (approximately, 100° to 120°) due to the increment of the swelling of hydrogel compartment. In addition, the hydrogel compartment is thicker in pH 4 than in DI water.

Thus, in certain aspects, the present disclosure provides a multiphasic micro-component, where prior to the substantial physical transformation, deformation, or reconfiguration, the micro-component has a first shape selected from the group consisting of: spheres, ovals, rectangles, polygons, disks, toroids, ellipsoids, cones, pyramids, rods, cylinders, and fibers. After the substantial physical transformation, deformation, or reconfiguration the micro-component has a second shape, distinct from the first shape, selected from the group consisting of spheres, ovals, rectangles, polygons, disks, toroids, ellipsoids, cones, pyramids, rods, cylinders, and fibers. In certain aspects, such a substantial physical transformation, deformation, or reconfiguration is substantially reversible, so that the multiphasic micro-component has an initial first state (e.g., a cylindrical shape in the example above) and after the presence of or the change in the external stimulus (e.g., ultrasound, pH, presence of a chemical like a solvent), the multiphasic micro-component has an altered second state (e.g., a spherical shape). The multiphasic micro-component returns to its initial state (e.g., to a cylindrical shape) after the external stimulus (e.g., ultrasonication, pH, presence of a solvent) is removed. Alternately, the multiphasic micro-component may similarly return to its initial state (e.g., to a cylindrical shape) after the external stimulus (e.g., ultrasonication) is returned to an initial level. In certain variations, prior to the substantial physical deformation, one phase or compartment of the micro-component has a first volume and after the substantial physical deformation, the phase or compartment of micro-component has a second volume distinct from the first volume. In other variations, prior to the substantial physical deformation, the micro-component has a first volume and after the substantial physical deformation, the micro-component has a second volume distinct from the first volume.

Reconfigurability is an important property of natural particles, such as spores or viruses, yet the experimental realization of reconfigurable designer particles remains by-and-large elusive. In accordance with present teachings, synthesis and dynamic reconfigurability of a novel type of multiphasic or multicompartmental microcylinders with anisotropic or isotropic mechanical responses are provided. Exposure of these microcylinders to an external stimulus, such as ultrasound or an appropriate solvent, gives rise to interfacial stresses that ultimately causes mechanical reconfiguration. The compartmentalized microcylinders can be prepared by electrohydrodynamic co-jetting of two or more polymer solutions to create bundles of aligned fibers with distinct compartments, which are finally microsectioned into multicompartmental microcylinders. Depending on the composition of the phases (e.g., microcompartment materials), these microcylinders undergo programmable shape-shifting of individual compartments or entire particles upon exposure to ultrasound. If the microcylinders are comprised of polymer phases or compartments with distinct swellability, reversible two-way shape-switching occurs, e.g., as the solvent environment is altered. Fully reversible three-way shape-toggling is also contemplated for biphasic microcylinders or other multiphasic microcomponents in general. Such three-way shape-toggling can occur in embodiments of biphasic microcylinders where a first phase or compartment comprises a hydrogel and a second phase comprises an organogel. Thus, the multiphasic/multicompartmental microcylinders made by electrohydrodynamic co-jetting can undergo active responses, such as shape-shifting, reversible switching, or three-way toggling. These active responses are hallmarks of biological systems, but prior to the inventive technology have not yet been systematically realized in synthetic colloidal materials. Imparting structural anisotropy through electrohydrodynamic co-jetting is potentially a rather generic materials processing strategy that enables a wide range of dynamically reconfigurable microcylinders with prospective applications as sensors, re-programmable microactuators, targeted drug delivery, or reversible self-assembly.

The precise engineering of particle properties is important for many biomedical applications including self-assembly, drug delivery and medical diagnostics. Beyond particle chemistry, physical properties, such as size, shape, and compartmentalization have been identified as key attributes that govern particle fate in contact with biological systems. While it has been attempted to create biological particles that mimic the complexity of biological particles, yet the these particles generally are not capable of reliably undergoing spontaneous reconfiguration. In contrast, nature's particles, such as spores, viruses or cells, can rapidly alter major phenomenological attributes, such as shape, size, or mechanical properties in response to environmental changes. The various multiphasic microcomponents of the present teachings provide synthetic analogues of nature's particles that can spontaneously reconfigure in response to defined pre-determined external stimuli. The present disclosure contemplates multiphasic microcomponents that are responsive to an external stimulus and reconfigure. Such microcomponents designed in accordance with the present teachings are capable of both irreversible, as well as reversible reconfiguration of microcylinders with anisotropic compartmentalization.

FIGS. 13A to 13C thus illustrate the preparation of PLGA microcylinders using electrohydrodynamic co-jetting and subsequent microsectioning, as described above. As shown in FIG. 13D, large populations of microcylinders having substantially the same diameters and well-defined and controllable length are obtained. Moreover, microcylinders with multiple, different compartments are prepared using a range of different needle sets including core/shell and dual-core/shell arrangements (FIG. 13A). If a PLGA sacrificial shell stream is employed during electrohydrodynamic co-jetting, simultaneous processing of substantially dissimilar materials is possible and allows for a broad diversification of the compartment compositions. Subsequent removal of the sacrificial shell results in microcylinders with substantially different compositions in the different phases or compartments. Selected target structures and their potential reconfigurability are shown in FIG. 13E.

For polymer particles, the thermodynamically most favorable state is that of a sphere and most particle fabrication methods have resulted in spherical polymer particles. Thus, most synthetic polymer particles have exclusively a spherical shape. If these microcylinders are comprised of polymers below their glass transition temperature, their shapes are arrested in the cylindrical shape and the particles stable over extended times. For example,

where PLGA is used as a biodegradable polymer, it has a T_{g} of 47-48° C., and a relatively low surface-tension (The water/air contact angle of a film cast from the PLGA is observed to be 92°). FIG. 18B shows a population of PLGA-based microcylinders, which are stored below the glass transition temperature of the polymer for 3 days (T_{g}>T_{P} regimen; where T_{g}: glass transition temperature and T_{P}: polymer temperature). To induce shape-shifting, either the T_{P} can be increased or T_{g} lowered. While direct heating of the particles for 24 hrs at 60° C. does not appear to result in detectable changes in particle shapes, ultrasound treatment for 2 minutes in water converts the microcylinders completely and homogenously into spheres (FIG. 18C). In certain embodiments, the external stimulus is optionally selected to be application of ultrasound energy. The choice of ultrasound as the stimulus for shape-reconfiguration comes with a number of potential advantages, as it can be applied remotely and has already found broad usage in medical and non-destructive imaging.

If the microcylinders are treated with ultrasound in an ice bath, the localized heating effect of the ultrasound is balanced and shape-shifting is no longer observed (FIG. 18D). In such a circumstance, the particles remain in the T_{g}>T_{P} regimen and their initial cylindrical shape is maintained. Ultrasound-mediated cavitation, as well as other mechanical effects, are ruled out as the driving force for shape-shifting, because they should be approximately temperature-independent. For a particle above its glass transition temperature, shape-shifting is favored by higher free surface energy of the particle surface and lower polymer viscosity. Assuming a close-to-constant polymer viscosity, the driving force for particle reconfiguration should be dominated by minimization of the free surface energy. Thus, it is believed that the shape-shifting appears to depend on the surface tension of the solvent, in which the reconfiguration is carried out. For confirmation, PLGA microcylinders are suspended in either apolar heptane (∈=1.9) or polar water (∈=80). After treatment with ultrasound for 2 min, particles undergo shape-shifting in water, but not in heptane. This finding confirms that minimization of free surface energy of the polymer particles plays a dominating factor for particle reconfiguration (FIG. 18E).

The present teachings also contemplate shape-shifting for microcylinders with multiple distinct compartments (FIG. 14A). Specifically, PLGA microcylinders with an average aspect ratio of 1.48+/−0.10 and two equally-sized compartments are prepared and exposed to ultrasound for 3 min (FIGS. 14B to 14D). The average diameter after shape-shifting was 26.85+/−0.88 μm as compared to an average diameter of 20.06+/−0.56 μm for the microcylinders prior to shape-shifting. The mean aspect ratio of the microcylinders changes from 1.48+/−0.10 to 1.05+/−0.03, which is indicative of near-to-perfect spheres. In addition, CLSM analysis after shape-shifting confirms maintenance of well-defined, bicompartmental particle architectures.

To further demonstrate the widespread applicability of the inventive shape-shifting approach to a variety of distinct multiphasic particles, bicompartmental microcylinders with an average diameter and length of 20.69+/−0.69 μm and 70.00+/−0.88 μm are prepared (FIG. 14E). Subsequent shape-shifting results in microspheres with diameters of 37.17+/−1.22 μm and aspect ratios of 1.02+/−0.01 (FIG. 14F). Again, the bicompartmental architecture of the particles is fully maintained. Beyond bicompartmental architectures, one may expect higher propensity for inhomogeneities during shape-shifting, as the number of compartments increases or the size of individual compartments decreases. Two types of tricompartmental microcylinders: those with sequential (FIG. 14G) and those with pie-shaped compartmentalization (FIG. 19A). As shown in FIGS. 14H and 19B, CLSM analysis of the compartmentalized particles confirms that the original compartmentalization patterns are fully maintained after shape-shifting. Similarly, heptacompartmental particles are accessible by shape-shifting of corresponding microcylinders. The synthesis of various multiphasic/multicompartmental microspheres via electrojetting can be used to create a broad range of compartmentalized microspheres capable of shape-shifting (FIGS. 19C-19D).

Furthermore, in certain aspects, the shape-shifting or reconfiguration can be limited to only select phases/compartments in the microcomponent. One of the major advantages of using compartmentalized microcylinders for shape-shifting is that appropriate selection of the phase materials permits partial shape-shifting, so that only certain phases/compartments of the microcylinders undergo shape-shifting (e.g., are converted into spheres), while the rest remains unaltered. Such partial shape reconfiguration provides colloidal particles with entirely new shapes. Microcylinders with compartments that feature polymers with distinct T_{g}, plasticizability or rigidity are thus employed. Here, poly(methyl methacrylate) (PMMA) and PLGA are used for electrohydrodynamic co-jetting, because PMMA has a T_{g} of 115-116° C., which is substantially higher than that of PLGA (47-48° C.). Heating the bicompartmental microcylinders to a temperature between the two glass transition temperatures results in selective shape-shifting of the PLGA compartment only (FIGS. 15A to 15D). Using this approach, a wide range of particle shapes are formed through programmable reconfiguration of microcylinders with different compartment geometries and/or polymer compositions. FIG. 15E shows four examples of reconfigured polymer particles (corresponding confocal images are provided in FIG. 20). Changes in the aspect ratios of bicompartmental PLGA/PMMA particles yields “bull-head” or “ring” particles. When the weight percentage of PMMA is decreased from 100% (FIGS. 15B to 15D) to 10% in one compartment (FIG. 15E), particles undergo increasing bending, which may be attributed to a decreased rigidity of the particles.

Other embodiments of bicompartmental particles are prepared with poly(vinyl cinnamate) (PVCi) confined in one phase or compartment. Because PVCi is a photocrosslinkable polymer, exposure to UV light can be used to render the PVCi containing compartments inert to the ultrasound-induced reconfiguration (FIG. 15E). The observed particle morphologies closely resemble theoretically predicted equilibrium shapes (FIG. 21). Specifically, the simulated equilibrium states for bicompartmental PLGA/PLGA (FIGS. 21A-21C) and PVCi/PLGA microcylinders (FIGS. 21D-21F) are in excellent agreement with the experimentally observed particle shapes. Similarly, tricompartmental PLGA/(PLGA+PVCi)/PLGA cylinders are reconfigured into spherical particles with centrosymmetric polymer backbones (FIG. 15E).

The particle embodiments described just above typically undergo on-way reconfiguration, in other word, their shapes are moving towards a thermodynamic equilibrium envelope. In other embodiments, however, multicompartmental microcylinders can undergo fully reversible and controllable two-way switching, as illustrated in FIGS. 16A and 16D. In FIG. 16A, a PLGA solution is used as a sacrificial shell stream and a hydrogel was processed through the core. FIG. 16B displays a representative SEM image of bicompartmental hydrogel/PLGA microcylinders after electrospinning, thermal crosslinking and microsectioning. The thermal crosslinking is employed to avoid dissolution of the core polymer in water. The hydrogel core compartment is swollen by 280% in water, while the PLGA compartment maintains its initial shape. The mismatch in mechanical properties causes reversible expansion and retraction of the hydrogel neck upon exposure to water (FIG. 16C). Moreover, the synthesis of bicompartmental microcylinders with phases comprising PVCi and PEO in a side-by-side arrangement leads to reversible bending of the microcylinders upon immersion into water (FIG. 16F). After crosslinking, the PVCi is swellable in dioxane, whereas the PEO compartment maintains the original shape in dioxane, resulting in reversible bending (FIG. 16F). Highly repeatable, reversible reconfiguration is observed under these conditions (FIG. 29A) with bending variabilities below 10% (FIG. 29).

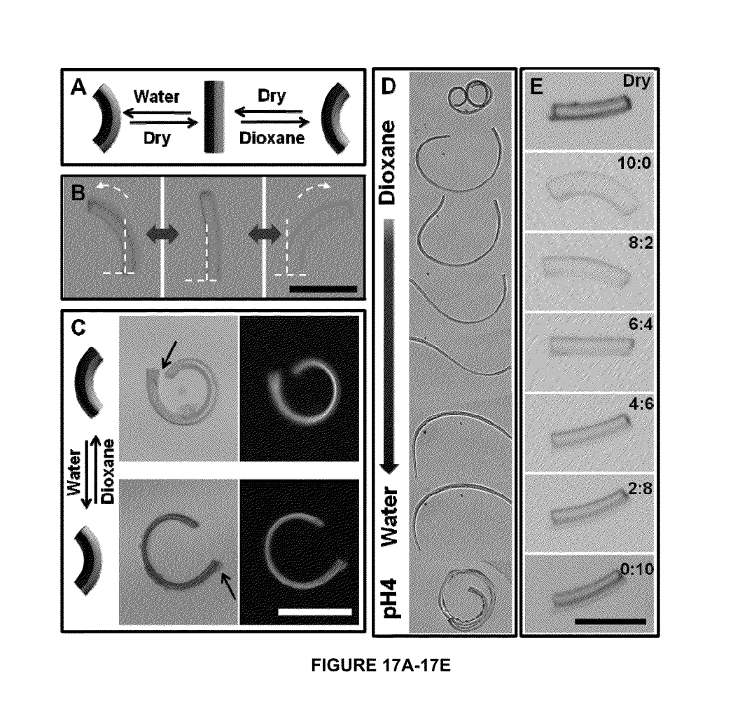

Fully reversible toggling is also contemplated for certain variations of the present teachings. For example, in an embodiment having bicompartmental microcylinders composed of two different stimuli responsive gel compartments (FIG. 17A). Experimentally, bicompartmental microcylinders configured as a pair of an organogel (e.g., PVCi) and a hydrogel (e.g., a chemically crosslinked 1:1-mixture of PEI/PEO) are prepared and evaluated for their mechanical actuation upon exposure to either dioxane or water (FIG. 17B). In dioxane, the PVCi compartment is selectively swollen, whereas the hydrogel compartment expands in water. In the dry state, however, both compartments are contracted and the cylinders assume a straight shape. For 200 μm fibers, closed circles are formed by swelling in dioxane (FIG. 17C). With longer fibers, helical structures are obtained in dioxane, where the PVCi compartment is located in the outside rim of the helix (top-most in FIG. 17D). Subsequent immersion into water results in reconfiguration of the helices into straight fibers. At pH 4, the hydrogel compartments selectively swell, resulting in a helical configuration (bottom-most in FIG. 17D. The actuation angle of the bicompartmental microcylinders successively varies with the solvent ratio of dioxane and water leading to a continuous transition from concave to convex (FIG. 17E).

In summary, the present teachings provide multicompartmental microcylinders with appropriately designed compartments that can undergo defined shape reconfiguration, such as toggling, bending or shape-shifting. An interesting aspect of these multicompartmental microcylinders is that different compartments can be made of different polymers that can selectively respond to a specific stimulus. If multicompartmental microcylinders are exposed to ultrasound as the external stimulus, irreversible one-way shape-shifting can lead to reconfiguration into near-to perfect spheres. Two-way, reversible shape transitions generally require multiphasic microcomponents that are comprised of stimulus-responsive, as well as inert phases/compartments. If the distinct phases or compartments of the microcomponents comprise a hydrogel and an organogel, the individual compartments display different swelling responses. This enables fully reversible three-way shape-toggling with well-defined convex-to-concave transitions. The controlled reconfiguration of multiphasic microcomponents, like microcylinders, constitutes important steps towards the development of dynamic materials with potential applications as sensors, actuators, or switchable drug delivery carriers.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A multiphasic micro-component capable of shape-shifting, the micro-component comprising a first phase and at least one additional phase distinct from said first phase, wherein one or more of said first phase and said at least one additional phase comprises a polymer and a component that is responsive to an external stimulus, so that the micro-component exhibits a substantial physical deformation in response to either: (i) the presence of the external stimulus or (ii) a change in level of the external stimulus.

2. The multiphasic micro-component of claim 1, wherein the external stimulus is selected from the group consisting of: temperature, pressure, light, pH, ionic strength, hydrophobicity/hydrophilicity, solvent, concentration, a stimulator chemical, sonic energy, electric energy, pressure, magnetic fields, and combinations thereof.

3. The multiphasic micro-component of claim 1, wherein the substantial physical deformation results in a change in shape, volume, or shape and volume, of at least one of said first phase and said at least one additional phase.

4. The multiphasic micro-component of claim 1, wherein prior to the substantial physical deformation, the micro-component has a first shape selected from the group consisting of: spheres, ovals, ellipsoids, rectangles, polygons, disks, toroids, cones, pyramids, rods, cylinders, and fibers, wherein after the substantial physical deformation the micro-component has a second shape distinct from said first shape selected from the group consisting of spheres, ovals, ellipsoids, rectangles, polygons, disks, toroids, cones, pyramids, rods, cylinders, and fibers.

5. The multiphasic micro-component of claim 1, wherein prior to the substantial physical deformation, the micro-component has a first volume and after the substantial physical deformation, the micro-component has a second volume distinct from said first volume.

6. The multiphasic micro-component of claim 1, wherein the substantial physical deformation is a substantially reversible deformation, so that the multiphasic micro-component has an initial first state and after the (i) the presence of the external stimulus or (ii) the change in the external stimulus, the multiphasic micro-component has an altered second state, wherein the multiphasic micro-component returns to its initial state after the external stimulus is removed or returned to its initial level.

7. The multiphasic micro-component of claim 1, wherein the component responsive to the external stimulus is the polymer.

8. The multiphasic micro-component of claim 7, wherein the first polymer responsive to the external stimulus is selected from the group consisting of: poly(lactide-co-glycolide) (PLGA), poly(vinyl cinnamate) (PVCi), and combinations thereof.

9. The multiphasic micro-component of claim 1, wherein at least one of said first phase and said at least one additional phase comprises an active ingredient.

10. The multiphasic micro-component of claim 9, wherein said active ingredient is selected from the group consisting of: a therapeutic active ingredient, a systemic active ingredient, a chemotherapy active ingredient, a localized active ingredient, an oral care active ingredient, a nutritional active ingredient, a personal care active ingredient, a cosmetic active ingredient, a diagnostic imaging indicator agent, and combinations thereof.

11. The multiphasic micro-component of claim 1, wherein the polymer is a pharmaceutically and/or cosmetically acceptable polymer selected from the group consisting of: biodegradable polymers, water soluble polymers, water dispersible polymers, water insoluble polymers, and combinations and co-polymers thereof.

12. The multiphasic micro-component of claim 11, wherein the pharmaceutically and/or cosmetically acceptable polymer is selected from the group consisting of: sodium polystyrene sulfonate (PSS), polyethers, polyethylene oxide (PEO), polyethylene imine (PEI), polylactic acid, polycaprolactone, polyglycolic acid, poly(lactide-co-glycolide polymer (PLGA), polyvinylpyrrolidone, hydroxyl alkyl cellulose, hydroxypropyl methyl cellulose (HPMC), hydroxypropyl cellulose (HPC), hydroxyethyl cellulose (HEC), methyl cellulose (MC), carboxymethyl cellulose (CMC), vinyl acetate, polyvinylpyrrolidone-vinyl acetate copolymers, polyvinyl alcohol (PVA), polyacrylates, polyacrylic acid (PAA), vinylcaprolactam/sodium acrylate polymers, methacrylates, poly(acryl amide-co-acrylic acid) (PAAm-co-AA), vinyl acetate, crotonic acid copolymers, polyacrylamide, polyethylene phosphonate, polybutene phosphonate, polystyrenes, polyvinylphosphonates, polyalkylenes, carboxy vinyl polymer, cellulose acetate, cellulose nitrate, ethylene-vinyl acetate copolymers, vinyl acetate homopolymers, ethyl cellulose, butyl cellulose, isopropyl cellulose, shellac, siloxanes, polydimethylsiloxane, polymethyl methacrylate (PMMA), cellulose acetate phthalate, natural or synthetic rubber; cellulose, polyethylene, polypropylene, polyesters, polyurethane, nylon, and copolymers, derivatives, and mixtures thereof.

13. The multiphasic micro-component of claim 1, wherein the first phase comprises a first polymer and the at least one additional phase comprises a second polymer distinct from the first polymer, wherein the first and second polymers are independently selected from the group consisting of: poly(lactide-co-glycolide) (PLGA), poly(vinyl cinnamate) (PVCi), poly(methyl methacrylate) (PMMA), poly(ethylene)oxide (PEO), and combinations thereof.

14. A method of controlling the shape of a micro-component, comprising:
exposing a multiphasic micro-component capable of substantial deformation to an external stimulus, wherein the multiphasic micro-component comprises a first phase and at least one additional phase distinct from said first phase, wherein at least one of said first phase and said at least one additional phase comprises a polymer and a component that is responsive to the external stimulus, wherein one or more of said first phase and said at least one additional phase exhibits a substantial deformation resulting in a change in shape, volume, or both shape and volume.

15. The method of claim 14, wherein the external stimulus is selected from the group consisting of: temperature, pressure, light, pH, ionic strength, hydrophobicity/hydrophilicity, solvent, concentration, a stimulator chemical, sonic energy, electric energy, pressure, magnetic fields, and combinations thereof.

16. The method of claim 14, wherein the exposing further comprises changing the external stimulus from a first level to a second distinct level.

17. The method of claim 14, wherein the exposing further comprises introducing the external stimulus to the multiphasic micro-component.

18. The method of claim 14, further comprising removing the external stimulus so that during the exposing of the external stimulus the multiphasic micro-component is deformed from a first state to a second distinct state and after said removing of the external stimulus, the multiphasic micro-component substantially returns to the first state resulting in a substantially reversible deformation of the multiphasic micro-component.

19. The method of claim 14, wherein prior to the substantially reversible deformation the micro-component has a first shape selected from the group consisting of: spheres, ovals, ellipsoids, rectangles, polygons, disks, toroids, cones, pyramids, rods, cylinders, and fibers, wherein after the substantially reversible deformation the micro-component has a second shape distinct from said first shape selected from spheres, ovals, ellipsoids, rectangles, polygons, disks, toroids, cones, pyramids, rods, cylinders, and fibers.

20. The method of claim 14, wherein prior to the substantially reversible deformation the micro-component has a first volume and after the substantially reversible deformation the micro-component has a second volume distinct from said first volume.

21. The method of claim 14, wherein the component responsive to the external stimulus is the polymer.

22. A multiphasic micro-component capable of shape-shifting, the micro-component comprising a first phase comprising a first polymer responsive to an external stimulus and at least one additional phase distinct from said first phase comprising a second polymer distinct from said first polymer, wherein the first phase exhibits a substantial physical deformation in response to: (i) the presence of the external stimulus or (ii) a change in the external stimulus, wherein the first and second polymers are independently selected from the group consisting of: poly(lactide-co-glycolide) (PLGA), poly(vinyl cinnamate) (PVCi), poly(methyl methacrylate) (PMMA), poly(ethylene) oxide (PEO), and combinations thereof.

23. A multiphasic micro-component capable of shape-toggling, the micro-component comprising a first phase comprising a first polymer responsive to a first external stimulus and at least one additional phase distinct from said first phase comprising a second polymer distinct from said first polymer and responsive to a second external stimulus, wherein the first phase exhibits a substantial physical deformation in response to: (i) the presence of the first external stimulus or (ii) a change in the first external stimulus, and the at least one additional phase exhibits a substantial physical deformation in response to: (i) the presence of the second external stimulus or (ii) a change in the second external stimulus.

24. The multiphasic micro-component of claim 23, further comprising a third phase distinct from said first phase and said second phase.

25. The multiphasic micro-component of claim 23, wherein the first phase comprises a hydrogel and the second phase comprises an organogel.
